(19) United States
(12) Patent Application Publication
(10) Pub. No.: US 2019/0189259 A1
(43) Pub. Date: Jun. 20, 2019

(54) SYSTEMS AND METHODS FOR GENERATING AN OPTIMIZED PATIENT TREATMENT EXPERIENCE

(71) Applicant: Gary Wayne Clark, Estes Park, CO (US)

(72) Inventor: Gary Wayne Clark, Estes Park, CO (US)

(21) Appl. No.: 16/228,495

(22) Filed: Dec. 20, 2018

Related U.S. Application Data
(60) Provisional application No. 62/608,351, filed on Dec. 20, 2017.
(60) Provisional application No. 62/654,669, filed on Apr. 9, 2018.

(51) Int. Cl. G16H 20/10; G16H 70/40; G16H 20/70; G16H 10/20; G16H 10/60

(52) U.S. Cl. CPC G16H 20/10; G16H 70/40; G16H 50/20; G16H 10/20; G16H 10/60; G16H 20/70

(57) ABSTRACT

Implementations described and claimed herein provide systems and methods for generating an optimized treatment experience for a patient. In one implementation, patient experience data is captured using at least one patient experience device. The patient experience data corresponds to a patient experience factor for the patient. A current level of the patient experience factor is determined using a patient experience processing system. The current level of the patient experience factor is determined based on the patient experience data. A customized therapy for the patient is generated based on the current level of the patient experience factor. The customized therapy is an alternative treatment to a drug therapy administration. An administration of a patient treatment experience is generated based on the customized therapy. The patient treatment experience is generated using the at least one patient experience device and includes one or more of patient sense stimulation and patient cognitive stimulation.

### CROSS-REFERENCE TO RELATED APPLICATIONS

The present application claims benefit under 35 U.S.C. § 119 to U.S. Provisional Patent Application No. 62/608,351, entitled “System and Method for Improved Patient Experience using Non-Drug Alternative Therapy(s) to Mitigate Pain and Stress While Lowering Drug Use” and filed on Dec. 20, 2017, and to U.S. Provisional Patent Application No. 62/654,669, entitled “System and Method for Improved Patient Experience using Non-Drug Alternative Therapy(s) to Positively Impact Pain, Stress, Anxiety, and Mobility While Lowering Drug Use” and filed on Apr. 9, 2018. Each of these applications is incorporated by reference in its entirety herein.

### FIELD

Aspects of the present disclosure relate generally to generating an optimized treatment experience for a patient and more particularly to managing patient experience factors, such as pain, stress, anxiety, depression, sleep, mobility, and/or the like, through a drug alternative therapy customized for the patient.

### BACKGROUND

Drugs are currently used to treat a variety of conditions, including pain, stress, anxiety, depression, sleep, mobility, and/or the like. However, use of drugs is often unnecessary to treat such conditions and frequently result in abuse. As a result, there is currently a drug epidemic in the United States. For example, a significant percentage of patients prescribed opioids for chronic pain misuse them, and both prescription and illicit use of opioids are the main driver of overdose deaths. During each of the past few years, opioids were involved in tens of thousands of deaths in the United States alone, with dozens of people dying each day from overdoses involving prescription opioids. With such an alarming rate of opioid and other drug related abuse and deaths, the human and economic costs are staggering.

Some patients are introduced to unnecessary drugs in a clinical setting, such as a hospital, medical provider facility (e.g., doctor, dentist, specialist, etc.), a behavior health facility, a mental health facility, and/or the like. Many such patients are introduced to the unnecessary drugs due to environmentally generated symptoms of patient treatment experience, such as non-therapeutic hospital noise, sensory overload, alarm fatigue, isolation, lack of information, lack of control, and/or the like, resulting in patients that are medicated or overmedicated for reasons unrelated to their health or treatment.

It is with these observations in mind, among others, that various aspects of the present disclosure were conceived and developed.

### SUMMARY

Implementations described and claimed herein address the foregoing problems by providing systems and methods for generating an optimized treatment experience for a patient. In one implementation, patient experience data is captured using at least one patient experience device. The patient experience data corresponds to a patient experience factor for the patient. A current level of the patient experience factor is determined using a patient experience processing system. The current level of the patient experience factor is determined based on the patient experience data. A customized therapy for the patient is generated based on the current level of the patient experience factor. The customized therapy is an alternative treatment to a drug therapy administration. An administration of a patient treatment experience is generated based on the customized therapy. The patient treatment experience is generated using the at least one patient experience device and includes one or more of patient sense stimulation and patient cognitive stimulation.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 is a block diagram of an example treatment experience system for generating an optimized treatment experience for a patient.

FIG. 2 is a block diagram of example patient experience components applicable to one or more aspects of the treatment experience system.

FIG. 3 illustrates example operations for generating an optimized treatment experience for a patient.

FIG. 4 shows an example experience user interface generated by the treatment experience system.

FIG. 5A depicts an example pain user interface generated by the treatment experience system.

FIG. 5B shows an example pain reporting user interface generated by the treatment experience system.

FIGS. 6A and 6B illustrate an example drug user interface generated by the treatment experience system.

FIG. 7 shows an example relax user interface generated by the treatment experience system.

FIG. 8 depicts an example sleep therapy user interface generated by the treatment experience system.

FIG. 9 depicts an example sound therapy user interface generated by the treatment experience system.

FIG. 10 shows an example smell therapy user interface generated by the treatment experience system.

FIG. 11 illustrates an example pain management user interface generated by the treatment experience system.

FIG. 12 depicts an example pain submission user interface generated by the treatment experience system.

FIGS. 13A and 13B show example pain log user interfaces generated by the treatment experience system.

FIG. 14 depicts an example customized therapy user interface generated by the treatment experience system.

FIG. 15 illustrates an example network environment for generating an optimized treatment experience for a patient.

FIG. 16 is block diagram of an example computing system that may implement various systems and methods discussed herein.

### DETAILED DESCRIPTION

Aspects of the presently disclosed technology relate to systems and methods for generating an optimized treatment experience for a patient. Generally, the systems and methods described herein involve a platform delivering non-drug alternative therapies for managing one or more patient experience factors, including pain, stress, anxiety, depression, sleep, mobility, and/or the like, via a treatment experience system with data analytics, machine learning and other artificial intelligence (AI) techniques. The platform captures patient experience data and biometric response data, including biometrics, mood, and vital signs of a patient as a patient treatment experience is administered pursuant to a library of curated alternative therapies, including, but not limited to, sound or music therapy.

Some aspects of the presently disclosed technology involve the introduction, tracking and individual self-directed optimization of a customized therapy through a patient treatment experience to discover appropriate drug-alternative therapy options and combinations for managing patient experience factors, without additional drugs or in combination with lowered drug dosages, by monitoring and providing biometric feedback. The patient treatment experience may include sense stimulation involving one or more of the five senses of the patient (sight, hearing, smell, taste, touch) and/or cognitive stimulation of the patient.

In one example aspect, based on a personal profile, the treatment experience system curates and tracks individual songs or other sounds for a particular patient as it monitors, captures, and analyzes biometrics, including vital signs. With biometric response data, the treatment experience system calculates and generates drug equivalent metadata with a non-drug therapeutic index that parallels prescription drug effectiveness on a scale of +/−1 to 10 for individual songs or other sounds as administered for the patient. The treatment experience system may utilize biometric response data, which may include self-reported responses, among other data, to guide the patient to discover, learn, and self-administer the exact music or other patient treatment experience of a customized therapy that delivers validated patient experience factor relief for the patient, in connection with an elimination or reduction of drug therapy. In one aspect, among other advantages, the treatment experience system discovers and generates a non-drug therapeutic index for pain, stress, anxiety, mobility, and/or other patient experience factor management by using customized drug-alternative therapies for generating a patient treatment experience that includes a curated catalog of patient specific treatment experiences, such as sound therapy, with the patient listening to a musical song, playlist or soundscape. By utilizing metadata, including processed therapy data and patient intelligence linked to the patient specific treatment experiences, a baseline (e.g., a sound therapy baseline) may be generated and administered to other patients or groups of patients having a similar patient or demographic profile.

The system and methods described herein improve patient healthcare by improving patient physical and mental health during treatment, while reducing or eliminating the reliance on drug therapy. By reducing or eliminating the reliance on drug therapy, chronic drug misuse and overdose is reduced, addressing the significant human and economic costs the population has historically suffered due to such misuse. The presently disclosed technology further improves computer technologies, which conventionally are provided as a one-size fits all approach, are provided irrespective of an impact on the patient, result in a sensory overload, increase or fail to address pain, stress, anxiety, depression, sleep, mobility, and/or other factors, among other issues. The systems and methods described herein provide an optimized treatment experience for a patient, among other advantages that will be apparent from the present disclosure.

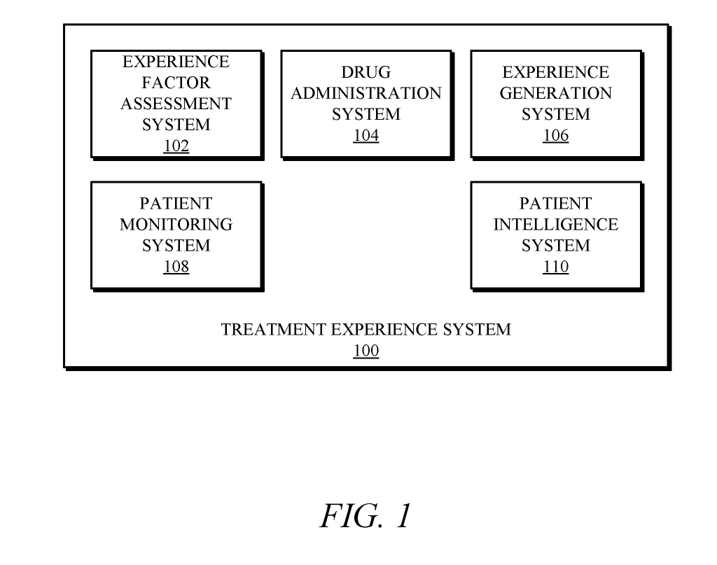

To begin a detailed description of an example treatment experience system 100 for generating an optimized treatment experience for a patient, reference is made to FIG. 1. In one implementation, the treatment experience system 100 includes an experience factor assessment system 102, a drug administration system 104, an experience generation system 106, a patient monitoring system 108, and a patient intelligence system 110. Each of these systems 102-110 may be separate from or integrated with each other in various combinations and include various structural components in a variety of combinations, as described herein.

In one implementation, the experience factor assessment system 102 captures patient experience data corresponding to at least one patient experience factor for a patient. The patient experience factors may include, pain, stress, anxiety, depression, sleep, mobility, and/or other factors that may impact patient physical or mental health or experience during a treatment. Generally, the patient experience factors may be directly treated through the experience generation system 106 in a complimentary or alternative medical approach to drug therapy, including prescription medications, such as pharmaceuticals, cannabis, and/or the like, as well as aggregation of biometric or biochemical projected templates of therapy for similar demographically profiled types of individuals or groups. To illustrate the presently disclosed systems and methods, pain is used as an example patient experience factor. However, it will be appreciated that such discussion is exemplary only and the presently disclosed technology is applicable to other patient experience factors.

The experience factor assessment system 102 may capture the patient experience data using a variety of techniques and devices as described herein. Further, the experience factor assessment system 102 may determine a current level of the patient experience factor using a variety of techniques based on the patient experience data. For example, the experience factor assessment system 102 may determine the current level of pain through self-reporting of pain levels of the patient on a numeric scale of 1 to 10, by inputs such as the patient responding to numeric prompts, pointing to or selecting a graphical series of facial expressions, pain-related body postures, facial recognition software enabled by a camera reading of micro expressions of the patient, body language, gestures, and/or facial expressions of the patient, and/or the like. In one implementation, the experience factor assessment system 102 utilizes an anatomical and descriptive principle where facial expressions are described in terms of a plurality of action units, which involve the unique changes produced by individual facial muscles or muscle combinations, and vocalized distress by moaning, crying or complaining, to determine a current pain level of the patient.

In one implementation, using the patient experience data, the experience factor assessment system 102 ranks the current level of the patient experience factor on a scale. With respect to pain, for example, the scale may include, without limitation, a numerical scale, a visual scale (e.g., the Wong-Baker FACES® pain rating scale), a global pain scale, a visual analog scale, a ladder, a McGill pain scale, a Mankoski pain scale, a color coded scale, a pediatric pain scale, a CPOT pain scale, and/or the like. In one implementation, the experience factor assessment system 102 prompts and interacts with the patient to create a customized pain scale particular to the patient. It will be appreciated that similar scales may be used for other patient experience factors.

The drug administration system 104, through supervision of a care professional, may evaluate the current level of the patient experience factor determined by the experience factor assessment system 102 and administer drugs at dosages and intervals accordingly and as needed. The drug administration system 104 communicates with the experience generation system 106 to generate a patient experience treatment that is a non-drug therapy in place of or complementary to the drug therapy administered by the drug administration system 104. As described herein, the treatment experience system 100 introduces the patient to alternative therapy choices with the experience generation system 106, provide biofeedback using the patient monitoring system 108, and determine updated levels of the patient experience factor using the experience factor assessment system 102 to manage the patient experience factor. The treatment experience system 100 builds a knowledge base using the patient intelligence system 110.

In one implementation, the experience factor assessment system 102 establishes a baseline and captures patient experience data for the patient experience factor. The patient experience data may be discovered, recorded and compared with historical levels over time to determine a current level of the patient experience factor. The current level of the patient experience factor may be determined by the experience factor assessment system 102, without limitation through: self-reporting by the patient, recorded via one or more patient experience devices, such as a Touch Screen, VUI, GUI or Haptic gesture; manually collection or reporting by a medical professional, caregiver, family member, or other authorized user; biometric readings; analysis, extraction, and/or capture from clinical records, such as an Electronic Medical Record [ERM] or Hospital Information System [HIS]; digital device interface software [DDI] from medical devices; analytically derived machine learning, metadata, sensors, biosensors, robots, ambient devices and artificial intelligence; and/or the like. In one implementation, the experience factor assessment system 102 utilizes enhanced facial emotion recognition software using multi-modal algorithms to analyze facial expressions, comparing real time and historical readings, such as multiple separate points on the face, eyebrows, eye corners, nose, mouth, head position and gaze for signs of depression, isolation, anxiety, neuropsychiatric disorders, somatoform disorders, unique changes produced by individual facial muscles or muscle combinations, and/or the like. The current level of the patient experience factor may be expressed, for example, as a numerical reading on a scale (e.g., 1-10), a visual depiction (e.g., a varying selection of emoticon faces), or through a historical comparison of self-reported, calculated or biometrically derived levels.

The drug administration system 104 captures patient drug data for the patient and determines a current drug administration level for the patient, which may be recorded and compared with historical drug administration levels over time. The drug administration system 104 may capture patient drug data from a variety of inputs; including, but not limited to: self-reporting by the Patient; manually collected by a medical professional, caregiver, family member, or other authorized party; computed by biometric readings; collected and disseminated from clinical records, such as an Electronic Medical Record [ERM] or Hospital Information System [HIS]; collected and disseminated by digital device interface software [DDI] from medical devices, such as an infusion pump; collected and reported by a medical implant device; collected and reported by a digital ingestion tracking system or smart capsule; collected and reported by a transdermal drug tracking system; collected and reported by a biochemical sensor or sensor activated wearable drug tracking system; collected and reported by a sensor activated drug tracking system connected to a sensor device, including, but not limited to a smartphone, smartwatch, wearable sensor, fitness monitor, tablet or computing device; analytically derived machine learning, metadata, sensors, robots, ambient devices and artificial intelligence; and/or the like.

The patient drug data may further include, without limitation: an amount and frequency by drug and dosage; interactions with additional drugs being administered to, or by patient; other biochemicals present in the samples, such as and hormone levels, such as epinephrine, norepinephrine, cortisol, dopamine, serotonin, tyrosine, tryptophan; and/or the like. The current drug administration level may be expressed, for example, as a numerical reading on a scale (e.g., 1-10), a visual depiction, or through a historical comparison of self-reported, calculated or biometrically derived levels.

Using the current level of the patient experience factor, alone or in combination with the current drug administration level, the experience generation system 106, generates a customized therapy for the patient that is an alternative to a further administration of drug therapy. The experience generation system 106 generates and administers a patient treatment experience based on the customized therapy. In one implementation, the patient treatment experience includes patient sense stimulation and/or cognitive stimulation.

In one implementation, the experience generation system 106 generates a patient treatment experience using sound therapy based on the customized therapy. The sound may include, without limitation, music therapy, noise cancelling soundscapes, sound replacement, musical genres, ambient soundscapes, natural soundscapes, meditative soundscapes, machine generated music, generative non-deterministic music, binaural beats, audio books, and recorded human vocal messages. The characteristics of the patient treatment experience, such as the sound of a music playlist, may be controlled and automatically tuned specifically for the patient by accessing the real time and historical biometric readings, vital signs, machine learning and/or AI. For example, as the patient begins listening to the sound of a music playlist, the tempo, or Beats per Minute [BPM] of the digital song may be recalibrated to match a biometric value of the patient in real time, such as heart rate. The digital song may then gradually slow down as it is played, by target changes, such as a preset target percentage or reduction in the patient experience factor is reached. The changes may be guided and controlled by the patient, provider, or other authorized user until reaching the target change or guided and controlled by machine learning or other AI algorithms that guide the patient to reach the target biometric value, such as heartrate or respiration, that reflect clinical best practices.

The experience generation system 106 may generate a patient treatment experience using sight therapy, smell therapy, touch therapy, taste therapy, and/or thought therapy based on the customized therapy. Sight therapy may include, for example, phototherapy, light emitting diodes, lasers, infrared light, light modulation, light therapy box, non-image forming light, artificial light that mimics natural daylight, helping stimulate serotonin production to alter moods by enhancing mood or sleep. Smell therapy may include aromatherapy, essential oils therapy, olfactory aromatherapy, massage aromatherapy, and/or the like. Touch therapy may include, without limitation, haptic therapy, massage therapy, physical therapy, chiropractic therapy, lasers, infrared light, acupressure, reike, reflexology, shiatsu, trigger point, therapeutic touch, acupressure, acupuncture, Swedish massage, rolfing, TENS, animal therapy, pet therapy, emotional support pet therapy and/or companion animal therapy, mobility therapy for Factors such as movement, balance and gait. Taste therapy may include, for example, Ayurveda therapy (six tastes or Rasas: sweet, sour, salty, bitter, pungent, and astringent). Further, taste therapy may be used in conjunction with the patient monitoring system 108 to identify taste and smell dysfunction that may be related to some other disease process that associated with several biochemical, metabolic or other pathologies, loss of function or distortion of function associated with fatigue or stress due to drug interactions. Thought therapy may include, cognitive behavior therapy, algiatry, mindful meditation, guided meditative audio and video programs, Tai Chi, Yoga, targeted breathing control with biofeedback, cognitive behavioral therapy, and/or the like.

It will be appreciated that the customized therapy may include one or more aggregations of therapies, including sense stimulation and/or cognitive stimulation. The experience generation system 106 analytically derives and targets the customized therapy for an individual patient using the patient experience generation system 106 and the patient intelligence system 110. The customized therapy executed by the treatment experience system 100 may be triggered by a sensor connected and/or ambient devices and software, animatronic life-like robots, ‘Carebots’, and other devices in various form factors, such as robotic humans, animals, toys, or other sensor connected devices, that monitor and detect changes in patient health, awareness and/or mood with their senses and thought as they impact patient experience factors. Examples of robotic devices that may be utilized for the treatment experience system 100 may be equipped with cameras to read facial expressions, sensors to capture vital signs, and speech recognition. Devices utilized by the treatment experience system 100 may meet the ISO 13482 standard for service robots.

The treatment experience system 100 may capture processed therapy data for analysis and storage in the patient intelligence system 110, including sound data, sight data, smell data, touch data, taste data, thought data, and other analytically derived machine learning, metadata, biochemical sensors, sensors, robots, ambient devices and artificial intelligence. The sound data may include playlists, songs, volume, duration, frequencies, wavelengths, modulation, pitch, and metadata, including, but not limited to: Genre, Origin, Era, Artist, Type, Tempo, Mood, Tempo, BPM, Keywords, and Metadata Source. The metadata may further include data generated by the treatment experience system 100 and/or patient interaction, machine learning, AI, which is extended metadata appended to the sound data and includes patient experience factor data, such as factor type (e.g. pain type), effectiveness in addressing the patient experience factor, demographic, patient profile (e.g., health and behavioral profile and preferences). This added metadata is an AI data driver for additional therapy discovery (e.g., music therapy) and curation that targets an individual and their confirmed biometric response to the patient experience treatment (e.g., music therapy) that has the most positive effect on the management of the patient experience factor. This AI driven metadata extension may also act as a personal music curator (or other therapy) guiding the patient to the most effective music for management of the patient experience factor. Sight data may include type, colors, wavelengths, modulation, intensity, duration, and metadata similar to that detailed above. Smell data may include type, intensity, metadata similar to that detailed above, and duration. Touch data may include type, intensity, metadata similar to that detailed above, and duration. Taste data may include type, intensity, metadata similar to that detailed above, and duration. Thought data may include type, intensity, metadata similar to that detailed above, and duration.

In one implementation, the patient monitoring system 108 captures real time biometric response data for the patient during the administration of the patient experience treatment by the experience generation system 106. The biometric response data may include biometric readings, vital signs, and/or the like. The biometric response data may be discovered, recorded and compared with historical readings over time for various patient experience treatments for the individual, including different sense stimulations and cognitive stimulations. Biometric response data may be used to adjust the delivery of the patient experience treatment and/or the underlying customized therapy. For example, the sound delivery of a music playlist may be controlled and automatically tuned specifically for an individual patient experience as the patient monitoring system 108 monitors the biometric response data and in response to other data captured in the patient intelligence system 110. In this example, as the patient begins listening to the sound of a music playlist, the starting tempo, or Beats per Minute [BPM] of the digital song, may be reset to match a biometric value of the patient, such as heart rate, in real time. The digital song may then gradually slow down as it is played, without degradation of either pitch or tonal quality, by a target change, such as a preset target percentage reduction, pursuant to care professional input, or by AI guided target changes that reflect clinical best practices.

The biometric response data may come from a variety of inputs; including, but not limited to: self-reporting by the patient; manually collected by a medical professionals, caregivers, family members, or other authorized party; collected and disseminated by medical devices, wearables, electronic tattoos, transdermal patches, implants, biometric sensors, biochemical sensors; using ‘Smart Home’ health and wellness sensors and monitors, including, but not limited to smart home hubs, radio frequency lightbulbs, motion, thermal imaging, sound, embedded optical facial recognition devices, speaker systems, images, air quality, temperature, humidity, hydration, light, weight, body movement, sleep patterns, heart rate, heart rate variability, blood pressure, respiration, facial ques, vocal tones, mood, bathroom events, carebots and medication monitors with embedded ambient intelligence machine learning, connected by the ‘Internet of Things’ [IoT], that are designed to collect health and wellness data related to selection, delivery and optimization of patient treatment experience; collected and disseminated from clinical records, such as an Electronic Medical Record [ERM] or Hospital Information System [HIS]; collected and disseminated by digital device interface software [DDI] from medical devices; and/or analytically derived machine learning, metadata, sensors, biochemical sensors, robots, ambient devices and artificial intelligence.

The biometric response data may include, without limitation, body temperature, galvanic skin response (gsr), heart rate, respiration rate, blood pressure, heart rate variability, muscle tension, peripheral oxygen saturation (SpO2), hydration, ECG signal, blood chemistry, and hormone levels, such as epinephrine, norepinephrine, cortisol, dopamine, serotonin, tyrosine, tryptophan; patient responsiveness measures such as mood, anxiety, awareness, motion, motor skills, cognitive and/or linguistic ability; voice analysis or recognition for stress or pain factors; a graphical depiction correlating biometric response data with patient experience factor levels, in real time, over time scales, representation within recommended ranges and aggregated demographic groupings. The experience factor assessment system 102 continues to capture patient experience data and update the current level of the patient experience factor and the customized therapy as biometric response data is captured and patient experience treatments are administered.

In one implementation, the patient intelligence system 110 gathers and ingests data into a knowledge base from the factor management for the patient, drugs, customized therapies, and biometric responses. Standard analytical views, queries, embedded and/or external educational content, complimented by AI machine learning, guides the patient and associated parties discover the optimal customized therapy that allow an individual patient to manage their patient experience factors while reducing the amount and frequency of drugs needed.

With regard to sound therapy, the treatment experience system 100, utilizes AI, machine learning and human curated music to guide the creation of music and sound playlist templates, by patient input or patient demographics and or social media data. These playlists may be optimized by the treatment experience system 100 for individual experience or aggregated as generic therapeutic starting points for patients, such as trauma and ICU environments. By mining patient demographics, social media, music preferences and/or family and caregiver input, combined with machine learning and predictive metadata analytics, the treatment experience system 100 guides the patient to discover an optimal individual experience with music in a therapeutic environment. As the treatment experience system 100 discovers, validates and curates efficacious management for patient experience factors, such as pain, by creating music templates for individuals, the patient intelligence system 110 will use big data analytics to extrapolate and refine algorithms and acoustic fingerprints to help other patients manage pain, lower stress, anxiety and increase mobility as they mitigate drug use by accessing the alternative therapies of music as complimentary medicine.

In some use cases, the intermittent or real time data monitored and analyzed from user biometrics and/or vital signs may be used by algorithms, machine learning and artificial intelligence to predict the onset of a patient episode (e.g., pain, stress, or anxiety). In this case, the user will be alerted of the impending episode and directed to access the most appropriate alternative therapy to ‘head off’ or lessen the episodic event.

In addition, the user interfaces and AI driven machine learning may collect information such as patient demographics, family history, medical history and Patient social media, music and personal experiences, or a sample set of sensory and cognitive stimulation for preferences as starting points to populate suggested customized therapy for patient discovery.

During the machine learning, the metadata for each customized therapy application will be collected, analyzed and matched with timestamped biometric response data for patterns that rank the effectiveness of the current therapy, creating a Non-Drug Therapeutic Index [NDTI] for each patient treatment experience, or combination thereof, with a + or − numerical impact on an appropriate scale for the patient treatment experience, such as the 1 to 10 scale of the Wong-Baker Faces Pain Ladder for each stimulation therapy applied with the system, such as an individual soundscape or song. The NDTI is then aggregated and applied to build a series of optimal NDTI scored playlists or soundscapes for the individual patient. For example, for the factor of music, music metadata collected, augmented and tracked by the NDTI will include, but is not limited to: Genre/Origin/Era/Artist/Type/Tempo/Mood/Tempo/BPM/Keywords/Metadata Source/Alternative Therapy Factor [for example, Pain, Stress, Anxiety, Mobility]/Alternative Therapy Effectiveness [NDTI].

As the patient experiences specific music playlists and songs, the treatment experience system 100 generates and adds indexed metadata for the patient's health condition, such as Pain Type/Pain Effectiveness, the NDTI and [+ or − 1 to 10] and Demographic Profile. This added metadata will then be used, along with other biometric data, to become the AI data driver for additional music discovery and curation that targets both the individual patient, and other similar patients with confirmed biometric or biochemical responses to music that has the most positive effect on their factor management. It is this AI driven metadata extension and non-drug therapeutic index [NDTI] that will also act as a personal music curator for factors such as pain, stress, anxiety and mobility that deliver the most effectiveness in the customized therapy, such as music.

AI and Machine Learning from the patient intelligence system 110 may also redirect and calibrate the characteristics of the patient experience treatment administration in real time [IRT] to accomplish the synchronization and control of the customized therapy, such as guiding cardiac rhythm by an external stimulus known as entrainment. For example, the characteristics of a soundscape or a song in a music playlist may be altered, controlled and/or automatically retuned in real time, specifically for an individual patient's experience, by accessing the real time and historical biometric response data, machine learning and/or AI algorithms. In this example, as the patient begins listening to the soundscape or song in a music playlist, the starting tempo, or Beats per Minute [BPM] of the digital song, may be reset to closely match a biometric value, such as heart rate or respiration rate, for the patient in real time. The digital soundscape or song may then be instructed by the treatment experience system 100 to gradually slow down as it is played, without degradation of either notation, volume, pitch or velocity by controlling the music using a music instrument digital interface [MIDI] controller.

In another example, the desired resulting target change may be to slow the patient's respiration rate down during the song by targeted percentage, such as 15%. The algorithm reads the patient's beginning heart rate of 86 beat per minute and sets the matching MIDI tempo of the song playback to match 86 BPM. The treatment experience system 100 determines that a 3-minute song and a target ending heart rate will result in the targeted reduction of 15%; computes that a 5% reduction in heart rate per minute will result in the targeted endpoint heartrate of 73 BPM; and recalibrates the MIDI tempo for song playback to gradually slow down over the 3 minute song to end at a tempo of 73 BPM. During the song playback, the treatment experience system 100 monitors the real time impact of the slowing MIDI tempo on the patient's biometrics, biochemicals and Vital Signs, and if the patient is not responding as desired, for example, the heart rate is not slowing down as desired, the treatment experience system 100 may reset the tempo to match the patient heart rate and start over trying to entrain a slowdown playback, with a new adjusted target. For example, based on the biometric response data, a 10% reduction is all that can be reasonably projected to be accomplished in this patient experience, so this is reset by the treatment experience system 100 as the new target for this instance of entrainment.

The treatment experience system 100 improves healthcare services and related technologies, including any underlying computer technologies, by optimizing the patient treatment experience, resulting in positive recovery and health for patients, lowered drug use and misuse, and reduced economic and human casualty costs associated with healthcare and drug misuse. For example, patients may be introduced to unnecessary dosages of opioids as pain drugs in a clinical setting such as a hospital, dentist office or behavioral health facility, or due to environmentally generated symptoms, such as non-therapeutic hospital noise, sensory overload and increased stress and anxiety, resulting in patients that may be over medicated in ways unrelated to their cause, which in turn slows healing and lowers the quality of the patient experience.

In a hospital setting, reducing noise and sensory overload while offering patients non-drug alternative therapies combined with individually tailored bio-feedback mechanisms driven by machine learning that compliment sound clinical practices, patients gain additional real time knowledge, feedback and control of their environment, discovering both autonomy and empowerment opportunities to lower stress and anxiety, mitigate pain and reduce the use of pain management drugs and decrease the likelihood of drug abuse and future dependence. In addition to improving the sensory environment for the patient, their caregivers, nurses and doctors providing care, the resulting improved patient experience contributes positively to a significant portion of financial reimbursements and insurance payments for a growing number of institutions as they shift from fee for service care arrangements to population health and value based outcomes payment models.

In everyday life, pain, stress, anxiety and mobility can be overwhelming, but reliance on drugs can be even worse, with staggering human and economic costs: over 63,000 opioid deaths in 1 year and $55 Billion a year for health and social costs; anxiety is the number one mental health disorder in the US with $42 Billion a year for health and social costs; and pain and anxiety costs $100 Billion per year for 100 Million people in US alone. The treatment experience system 100 provides a natural alternative to drugs, recognizing that the right therapy, including music therapy for healing, is different for every person. The treatment experience system 100 utilizes non-drug therapy, targeted to each individual, to biometrically manage pain and anxiety, using less drugs or no drugs at all. Accessing proven alternative therapies for each of the five senses through sense stimulation and cognitive stimulation, patients measure their individual biometric responses to learn to select the specific alternative therapy(s) that help them control and reduce pain, stress, and anxiety, among other factors.

A hospital patient is typically exposed to hundreds of medical machine alarms per day; an ICU nurse over a hundred medical machine alarms per hour; and the average nurse must respond to over 3.7 medical machine alarms per minute. As noise levels in a clinical setting increase, sensory overload overwhelms both patients and caregivers; machine alarms are sometimes consciously or subconsciously ignored. The problem of sensory overload has grown to such proportions that ‘Alarm Fatigue’—not responding to a medical machine alarm—is now a recognized cause of death in hospitals. For patients exposed to intense sensory overload, such as increased sounds and constant medical machine alarms, the extraneous noise can alter memory, increase agitation and create feelings of isolation that lead to a lower tolerance for pain. Ironically, these environmentally generated symptoms can lead directly to an increase need for drugs, causing patients to be over medicated in ways unrelated to their cause.

Just as sensory overload contributes to feelings of isolation and less tolerance for pain, so does the lack of control and fear of the unknown in a clinical setting. Patient confusion and stress also increase anxiety, resulting in a lower tolerance for pain, and an increased need for prescribed drugs. By increasing the patient's knowledge and control over a stressful clinical environment, reducing the sensory overload, and introducing a guided selection of non-drug alternatives for pain mitigation, the treatment experience system 100 lowers pain, stress, and anxiety levels while increasing mobility to promote a better healing a therapeutic environment. Additionally, increasing patient autonomy and empowerment can help to lower pain, stress, and anxiety levels while increasing mobility and improving patient experience.

For example, when research studies compared the post-surgical morphine use for self-administered patient drug infusions between groups with access to meditative music, and other groups without access to music; the groups with access to music requested lower drug use and had a significantly higher likelihood of having self-reported pain relief than the subjects not exposed to music. There is a neurological basis for this finding, as researchers have found that listening to music releases the neurotransmitter dopamine in the brain, sending a pleasure signal to the rest of the body, allowing the brain to offset some of the need for prescribed drugs for pain mitigation. Music helps reduce pain by activating sensory pathways that compete with pain pathways, stimulating emotional responses, and engaging cognitive attention. Music, therefore, provided a cognitive distraction and meaningful intellectual and emotional engagement to help reduce pain. But not all music generates this positive chemical response—it must be matched to the individual's auditory receptivity for the optimal therapeutic effect.

Mind-body therapies are treatments that are meant to help the mind's ability to affect the functions and symptoms of the body. Complimentary medicine, or integrative medicine, when combined with traditional medicine, as many research studies have shown conclusively, can offer patients an improved mental state to heal faster, feel better about their clinical treatment and be discharged from a clinical setting sooner. The goal of integrative medicine is to maximize the wellness of the whole person, mind, body and spirit, not just an underlying disease or symptom, such as pain, stress, anxiety, or lack of mobility. This can be accomplished by combining the best of traditional medicine with the best of less conventional practices, alternative therapies that have a reasonable amount of high-quality evidence to support their use.

Researchers and health care professionals are finding that integrative medicine can provide positive outcomes for a broad range of pain, stress, anxiety and decreased mobility causes. This is because pain is often a whole-body experience; pain doesn't always come from just one source. There's the physical cause of the pain, of course, such as the injury, the joint pain, the muscle strain. But this physical pain can often be compounded by stress, anxiety, frustration, fatigue, decreased mobility plus the medication side effects combined with many other factors. Conventional medicine typically only addresses physical pain. This is where integrative medicine therapies can step in, to help with myriad other factors associated with pain. By using mind-body alternative therapies and introducing cognitive distractions that tap into the patient's senses of sound, sight, smell, touch, taste and thought, the treatment experience system 100 improve the patient's experience and mental state while offering the advantage of low cost, ease of provision, and safety for effective factors of pain, stress, anxiety, depression, sleep and mobility management programs. In summary, improving the patient experience by stimulating the five senses and cognitive thought improves healthcare and related technologies, while reducing costs drug dependency and improving patient health and treatment experience.

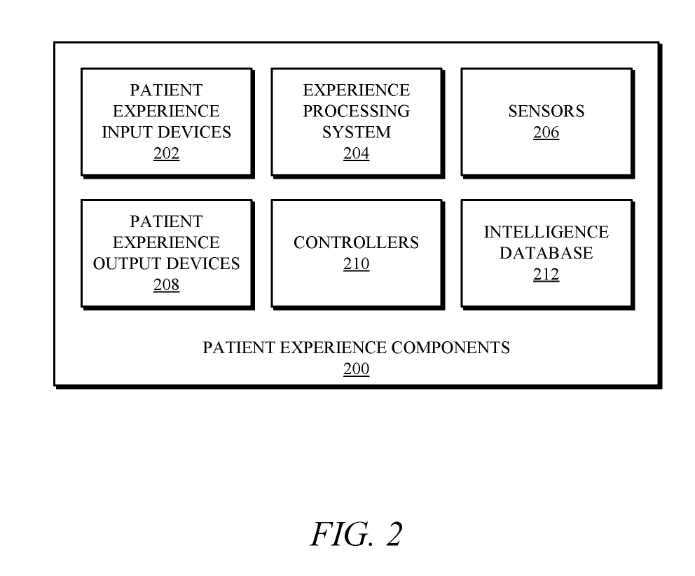

Referring to FIG. 2, example patient experience components 200 applicable to one or more aspects of the treatment experience system 100 are illustrated. It will be appreciated that each of the patient treatment components 200 may be used, alone or combination, by one or more of the systems 102-110 and may be separate from or integrated with each other in one or more systems and/or devices. In one implementation, the patient treatment components 200 includes one or more experience input devices 202, an experience processing system 204, one or more sensors 206, one or more experience output devices 208, one or more controllers 210, and an intelligence database 212. Patient experience devices described herein may include one or more of the experience input devices 202, the experience output devices 208, the sensors 206, and/or the controllers 210.

In one implementation, the experience input devices 202 includes or generates using a computing device(s), without limitation, an interface that accesses data for patient vital signs; an interface that accesses data for drug infusion pumps; an interface with clinical records, such as an Electronic Medical Record [ERM] or Hospital Information System [HIS], that accesses data for patient information; an interface that accesses data [DDI] from various medical devices; an interface with that accesses data from patient centric medical devices, wellness and fitness monitors, such as wearables (e.g., sensors or devices worn by, positioned on, or touching the patient), hearables (e.g., biometric enabled headphones with sensors), lickables (e.g., biochemical enabled sensors), transdermal patches, implants, biometric sensors, biochemical sensors, medication compliance monitors; ‘Smart Home’ health and wellness sensors, monitors, biosensors and biochemical sensors, including, but not limited to smart home hubs, radio frequency lightbulbs, motion, thermal imaging, sound, embedded optical facial recognition devices, speaker systems, images, air quality, temperature, humidity, hydration, light, weight, body movement, sleep patterns, heart rate, heart rate variability, blood pressure, respiration, facial ques, micro expressions, vocal tones, mood, bathroom events, carebots and medication monitors with embedded ambient intelligence machine learning, connected by the ‘Internet of Things’ [IoT] devices that are designed to collect health, wellness and fitness data related to selection, delivery and optimization of customized therapy; an interface rules engine of logic, algorithms and machine learning designed to collect, monitor, analyze, filter and distribute all information relevant to a patient experience in a clinical or outpatient setting; including, but not limited to clinical data, alarm management, location, and clinical research relevant to a Patient's pain management and sensory control, such as stress levels, vital signs, and alternative therapies applied for complementary medicine with the treatment experience system 100; analytically derived machine learning, metadata, sensors, bio sensors, biochemical sensors, robots, ambient devices and artificial intelligence.

In one example, the experience processing system 204 includes, but is not limited to: one or more printed circuit boards; one or more processing units; one or more data ports; one or more charging ports; one or more chargers from traditional power sources, or user generated power including, but not limited to kinetic, biochemical, or biomechanical generation; one or more power sources; one or more communication modules for wired or wireless communication, such as Bluetooth XLE or 5G wireless technology; one or more memory or storage modules; memory for storage of programs, algorithms and data; one or more user interface [UI] software modules, for driving user interfaces such as a voice user interface [VUI], a graphical user interface [GUI], a touch screen interface [TS], an audio user interface [AUI], a Virtual Reality [VR] and/or Augmented or Mixed Reality [AR] interface, a holographic user interface [HUI], a Virtual AI Assistant(s) [VA] interface; an artificial intelligence [AI] machine-learning module for optimization of inputs, processing and outputs; and/or the like. The experience processing system 204 may be hosted and/or spread across a network of central, edge, cloud, hybrid, mobile, IoT, virtual or embedded systems and methods, including, but not limited to infrastructure as a service [IaaS], platform as a service [PaaS], software as a service [SaaS] models, using either HIPPA secure transactions and/or a healthcare blockchain technology. If patient healthcare data is stored in a HIPPA compliant and/or patient owned blockchain, both data and trust will allow access, authentication and data sharing quickly and securely across the healthcare spectrum.

In one implementation, the sensors 206, the experience input devices 202, and/or the output experience devices 208 include, without limitation, one or more accelerometers, for measuring acceleration associated with a change in movement; one or more EMP HAPTIC actuators, for conveying information to a patient with vibration patterns and waveforms; one or more HAPTIC Sensors, for detecting patient vibration patterns, waveforms and interaction; one or more gyroscopes, for measuring orientation based on the principle of rotation; taking into account weight, shape and speed; one or more spectrometers, including near infrared spectrometers and molecular spectroscopy, for measuring environmental factors; one or more biochemical sensors, for measuring changes in user biometrics and various biochemicals, including, but not limited to epinephrine, norepinephrine, cortisol, dopamine, serotonin, tyrosine and tryptophan; one or more light sensors, for detecting brightness and color wavelengths, spectrums and hues; one or more light emitting diodes, for generating varying color wavelengths and brightness; one or more heating units, for olfactory diffusion of therapeutic oils and/or substances; one or more aroma plug-in modules, for modular replacement of therapeutic oils and/or substances; one or more taste plug-in modules for modular replacement of therapeutic tasting oils and/or substances; one or more clocks; one or more microphones and audio inputs; one or more speakers and audio outputs; one or more video inputs and outputs; one or more data ports; one or more charging ports; one or more chargers from power sources, or user generated power including, but not limited to kinetic, biochemical, or biomechanical generation; one or more batteries; one or more communication modules for wired or wireless communication, such as Bluetooth XLE or 5G wireless technology; one or more memory or storage devices; analytically derived machine learning, metadata, sensors, robots, ambient devices and artificial intelligence; and/or the like.

The output experience devices 206 includes or generates using a computing device(s), without limitation: one or more touch screen interface [TS], providing interactive pressure sensitive information display, holographic, or virtual surface using resistive, surface wave, capacitive or other interactive modalities; one or more voice user interfaces [VUI], providing mixed initiative dialogs and real time multilingual translation, with the language being changeable and selectable by the patient or other user or otherwise automatically detected; one or more graphical user interfaces [GUI], providing programs that allow graphical control of information by a user by selection of a mouse, keyboard, touch screen, voice or optical control by tracking of eye movements; one or more audio interfaces [AUI], providing audio detection and transmission of sounds and information to one or more listeners, from speakers, cochlear implants, assisted listening devices [ALDs], augmented and alternative communication devices [AAC], alerting devices [AD]; one or more Virtual Reality [VR] and/or Augmented or Mixed Reality [AR] interfaces, providing an interactive computer generated or computer augmented 3D user reality for background and foreground presentation using techniques such as video, audio, motion, eye movement, gestures, haptics, and/or other biometric or human initiated inputs or reactions for the detection and transmission of information; one or more holographic user interfaces [HUI], providing for the detection and transmission of information to manipulate a computer-generated 3D desktop using a Wii-like remote control, pointing at dialog boxes and icons hovering in the air and clicking on them rather than moving a mouse pointer on a 2D screen; virtual AI Assistant(s) [VA], providing an interactive conversational cognitive personification for human-interfacing artificial intelligence, machine learning and information discovery and display for a variety of user roles, such as Patients, nurses, doctors, caregivers, researchers and family members; an interface with clinical records, such as an Electronic Medical Record [ERM] or Hospital Information System [HIS], to retrieve and/or publish information for a variety of user roles, such as Patients, nurses, doctors, caregivers, researchers and family members; an input system for collecting patient responsiveness measures such as mood, stress, pain anxiety, awareness, depression, isolation, motion, mobility, cognitive, motor, and/or linguistic ability; and/or the like.

In one implementation, the experience processing system 204 generates or provides: an interface for information collected and disseminated from clinical records, such as an Electronic Medical Record [EMR] or Hospital Information System [HIS] and information analytically derived from machine learning and artificial intelligence algorithms; an interface for information collected and disseminated by digital device interface software [DDI] from medical devices, such as infusion pumps [PCA], drug delivery systems, medication monitors, medical alerts and alarms; a vital signs interface, for collecting patient information such as heart rate, heart rate variability, muscle tension, blood pressure, respiration, peripheral oxygen saturation (SpO2), hydration, ECG signal, blood chemistry, and hormone levels, such as epinephrine, norepinephrine, cortisol, dopamine, serotonin, tyrosine, tryptophan; an interface for information collected and disseminated by medical devices, wearables, electronic tattoos, transdermal patches, implants, biometric sensors; ‘Smart Home’ health and wellness sensors and monitors, including, but not limited to smart home hubs, radio frequency lightbulbs, motion, thermal imaging, sound, embedded optical facial recognition devices, speaker systems, images, air quality, temperature, humidity, hydration, light, weight, body movement, sleep patterns, heart rate, heart rate variability, blood pressure, respiration, facial ques, micro expressions, vocal tones, mood, bathroom events, carebots and medication monitors with embedded ambient intelligence machine learning, connected by the ‘Internet of Things’ [IoT], that are designed to collect health and wellness data related to selection, delivery and optimization of customized therapy; and an interface with analytically derived machine learning, metadata, sensors, robots, ambient devices and artificial intelligence.

In one implementation, the controllers 210 include various input and/or output devices, which are either wired or wirelessly connected for communication, processing and feedback. The controllers 210 may include the experience input devices 202, the experience processing system 204, the sensors 206, the experience output devices 208, and/or any combination and/or standalone modality of the patient experience components 200 or aspects of the systems 102-110. Each type of the controllers 210 allows input output interactions for a variety of functions, including but not limited to patient self-administered drugs, customized therapy generation, patient treatment experience generation and administration, biometric response monitoring, patient monitoring, patient experience factor assessment, patient intelligence management, access, and generation, plus added controls for accessing the various aspects of the treatment experience system 100. The controllers 210 may include, without limitation: a single or multi button drug infusion pump style controller [PCA]; a single or multi button joystick style controller; a multimedia console controller; a smartphone; a user device; a touch screen controller for Graphical User Interfaces [GUI] for smartphones, smartwatches, wearables, hearables, lickables, tablets, laptops, computers, etc.; a voice activated controller Virtual AI Assistant(s) [VA]; an eye monitoring controller Holographic user interfaces [HUI]; a Virtual Reality [VR] and/or Augmented or Mixed Reality [AR] controller; a Voice user interfaces [VUI] controller with language selection control or automatic detection; an inanimate object embedded controller is embedded into form factors of familiar and friendly objects, such as a squishy ball, fuzzy animal, favorite toy, drumstick or baton; a haptic actuated glove controller with multiple haptic sensors, receptors and actuators; an audio user interface [AUI] for audio detection and transmission of sounds; a ‘lickable’ interface controller designed to detect real time changes in user biometrics, vital signs and biochemicals, including, but not limited to epinephrine, norepinephrine, cortisol, dopamine, serotonin, tyrosine and tryptophan detected during the use of patient treatment experience, such as music therapy, for a patient experience factor, such as pain or anxiety from a biochemical saliva sensor mounted on a form factor such as a flavored reusable lollypop with a disposable sensor film, with the biosensor detecting and recording the biochemical levels in saliva, storing the values on a PCB, and/or transmitting the values to the experience processing system 204. The controllers 210 and/or the treatment experience system 100 may utilize drug infusion, glove ball, and game driven interfaces; game console interfaces; robotic interfaces that can hold, be held, or touch the patient, among other features; a biochemical interface; a PCA infusion interface; and/or the like.

In one implementation, the controller 206 utilizes a biochemical interface to detect real time changes in user biometrics and various biochemicals, including, but not limited to epinephrine, norepinephrine, cortisol, dopamine, serotonin, tyrosine and tryptophan detected during an administration of a patient experience treatment from a bio sensor, a biochemical saliva sensor is mounted on a flavored lollypop with a disposable sensor film. The biosensor detects and records the biochemical levels in saliva, stores them on a PCB and then transmits the readings via a Bluetooth or 5G wireless technology link to the experience processing system 204. The flavor modules are disposable, and the rest of the controller unit may be reusable. This “lickable” controller provides; a reusable biochemical sensor with a Bluetooth or 5G wireless technology transmitter and disposable taste therapy flavor modules. The patient “licks” the disposable flavor taste therapy module on lollipop, the biosensor reads and stores the biochemical levels in the saliva real time on a PCB; the Bluetooth transmitter sends a wireless signal with the biochemical data to a smartphone, tablet, laptop or computer to build a record of the real time biochemical changes in coordination with the customized therapy being applied. This biochemical marker is one of the vital signs used to measure the effectiveness for a therapy, or combination therapies, impacting the non-drug alternative therapy index for the patient, and then allowing adjustments or changes to the therapies for the user to achieve desired therapeutic results. Real time biochemical levels, such as dopamine, may be displayed in connection with the patient treatment experience being or previously administered, such as a particular song or sound. Stated differently, the controller 206 provides for biochemical, such as dopamine, detection and mapping to a particular patient treatment experience therapy, such as a song, to determine an impact on patient experience factor mitigation or management, such as pain mitigation.

The PCA infusion interface for the controller 206 may include a thumb sensor to control drug administration and/or smell therapy, including setting time, duration, pressure, and frequency. The smell therapy may include aromatherapy, calming smells (e.g., lavender). The PCT infusion interface may further include haptic sensors and controllers to sense biometrics, such as heart rate, provide an experience treatment, such as a massage, along with controls for sleep, calling a care provider, controlling lights, audio, messages, videos, music, books, and/or other patient treatment experience therapies.

In one implementation, the controllers 206, experience output devices 208, and/or the like may generate output interfaces, including, but not limited to: an interface with clinical records, such as an Electronic Medical Record [ERM] or Hospital Information System [HIS]; activity logs of all patient activities and data, delivered via a variety of modalities, such as a touch screen [TS], graphical user interface [GUI], or multilingual voice user interface [VUI]; standard reports, delivered via a variety of modalities, such as a touch screen [TS], graphical user interface [GUI], or voice user interface [VUI]; non-standard queries and reports delivered via a variety of modalities, such as a touch screen [TS], graphical user interface [GUI], or voice user interface [VUI]; and/or the like.

The intelligence database 212 may ingest and store data, including processed therapy data, generated and captured by the patient intelligence system 110 and/or other aspects of the treatment experience system 100. The intelligence database 212 may include, without limitation, an interface with clinical records, such as an Electronic Medical Record [ERM] or Hospital Information System [HIS]; analytical views of patient experience data, drug administration data, biometric response data; customized therapies; effectiveness data, and other processed therapy data; machine learning predictive analytics to help to guide the patient and caregivers to discover the optimal use of customized therapies to assist in patient discovery to lower patient experience factor levels, and reduce the amount of drugs needed to mitigate the same; AI, machine learning and human curated music metadata that guides discovery and curation of playlist templates by patient input or patient demographics and/or social media data that can be optimized for individual experience or aggregated as generic starting points for Patients, such as trauma and ICU environments; storage of patient-generated health data [PGHD] including, but not limited to medical history, vital signs, patient experience factor levels, drug use, alternative therapy(s), biometrics, processed therapy data, machine learning and AI for individual optimization for managing pain, stress and drug use, among other factors, will be polled and stored using a HIPPA compliant process, which may include a healthcare blockchain functionality and once stored in a patient owned blockchain, both data and trust can be accessed, authenticated and shared both quickly and securely across the healthcare spectrum; and/or the like. The treatment experience system 100 provides: an interface or embedded educational content for discovery of methods, techniques, research and shared social experiences for optimizing customized therapy; an interface, system and methods to gather data to the knowledge base from the processed therapy data, standard analytical views, queries, embedded and/or external educational content, complimented by AI machine learning, to help guide the patient and caregivers to build templates that assist discovery of the optimal alternative therapies that allow an individual patient to manage patient experience factors and reduce the amount of Drugs needed to mitigate the same. In addition, the treatment experience system 100 may collect information such as patient demographics, family history, medical history and patient social media, music and personal experiences, or a sample set of sensory stimulation of smell, light, sound, touch, taste and thought for preferences as starting points and or templates to populate suggested customized therapies for patient discovery.

Processed therapy data obtained and/or managed by the patient intelligence system 110 illustrates that patient knowledge and individually selected patient treatment experiences corresponding to customized therapy can impact patient experience factors, reducing the need for drugs. Specific data may be collected for an individual patient, and when analyzed by the patient intelligence system 110, optimization of the most effective combination of complementary medicine, or integrative medicine, such as alternative therapy and traditional medicine will allow patient discovery of an improved mental state to heal faster, feel better about their clinical treatment, use less drugs and be discharged from their clinical setting sooner. In one example, summary information is captured and presented, documenting the clinical effects of sound by the therapeutic application of music, combined with noise cancelling soundscapes, to elicit positive mind/body responses, such as increasing the naturally generated dopamine in the patient's brain, raising their tolerance for pain, lowering their stress levels, and lowering the patient's need for drugs to mitigate pain. Data for the introduction of the alternative Therapy of Music, including, but not limited to, the specific genre, song, artist, mood, source, passage, volume, duration, recorded human vocal messages and relevant metadata are captured and stored in the intelligence database 212 along with the patient experience data, biometric response data, drug administration data, and processed therapy data.

By data mining patient demographics, social media and music preferences, combined with machine learning and predictive metadata analytics, the treatment experience system 100 guides the patient to discover an optimal individual experience with music in a clinical environment. As the treatment experience system 100 discovers, validates and curates efficacious and effective pain management music templates and other patient experience treatments for the individual, the knowledge base of the patient intelligence system 110 uses big data analytics to refine algorithms and acoustic fingerprints that might help others manage pain, lower stress and mitigate drug use by accessing the alternative therapy of specific music as complimentary medicine.

To aggregate general use templates, individual patient data is scrubbed of any unique patient specific identifiers in compliance with all applicable patient security and privacy guidelines, such as HIPPA, and then combined with additional aggregate patient therapy data to generate processed therapy data allowing the use of views, queries, rules based processing, algorithms and/or AI Machine Learning analysis, research and discovery of new and previously unknown combinations of complementary medicine, integrative medicine or alternative Therapy and traditional medicine to actively promote future patient discovery of individual ways to manage patient experience factors, lowering drug use and improving the patient experience by allowing the patient to positively control an aspect of their own therapeutic healing environment. AI and Machine Learning from the patient intelligence system 110 may redirect and constantly recalibrate the characteristics of the patient treatment experience administration, in a background mode or in real time. For example, the sound of a music playlist may be controlled and automatically retuned in real time specifically for an individual patient's experience by accessing the real time and historical biometric readings, vital signs, machine learning and/or AI algorithms. In this example, as the patient begins listening to the sound of a music playlist, the starting tempo, or Beats per Minute [BPM] of the digital song, may be reset by the computing device to match the biometric value for the patient, such as heart or respiration rate, and/or biomarkers that detect neurotransmitter imbalances for chemicals such as epinephrine, norepinephrine, cortisol, dopamine, serotonin, tyrosine and tryptophan levels, in real time or delayed presentation to the patient. The digital song or soundscape may then gradually slow down as it is played, without degradation of either notation, pitch, volume or velocity by controlling the music using a music instrument digital interface [MIDI] control, by a target change, such as a preset target percentage reduction, a change set by patient or provider input of desired target percentages, or by AI guided target percentages that reflect clinical best practices. In addition to dynamically setting the starting digital song tempo to attempt to guide the patient to a targeted heart or respiration rate, or in the case of neurotransmitter imbalances, targeted biochemical levels, the patient's real time biometric response data may indicate that the target change is not being achieved. At this point, guided by AI algorithms, the treatment experience system 100 may dynamically recalibrate the real time music playback tempo and the target change for the reset tempo to a more achievable level of overall compliance with biometric values, patient experience, and clinical best practices.

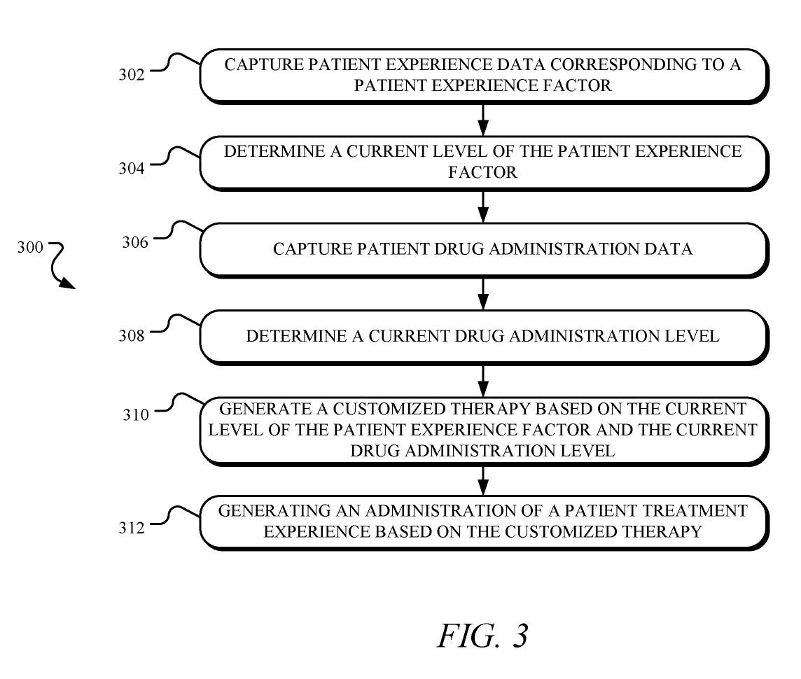

FIG. 3 illustrates example operations 300 for generating an optimized treatment experience for a patient. In one implementation, an operation 302 captures patient experience data corresponding to a patient experience factor. The patient experience data may be captured using at least one patient experience device and/or through at least one of: self-reporting by the patient, reporting by a medical professional, facial recognition, gesture recognition, analysis of patient records, or biometric readings. The patient experience factor may be at least one of pain, stress, anxiety, depression, sleep, or mobility. An operation 304 determines a current level of the patient experience factor using a patient experience processing system. The current level of the patient experience factor is determined based on the patient experience data. A patient episode may be based on the current level of the patient experience factor.

An operation 306 captures patient drug administration data, and an operation 308 determines a current drug administration level based on the patient drug data. The operation 306 may use at least one patient monitoring device to capture the patient drug administration data. For example, the at least one patient monitoring device may include a bio sensor configured to detect one or more biochemicals in saliva of the patient.

An operation 310 generates a customized therapy based on the current level of the patient experience factor and/or the current drug administration level. The customized therapy is an alternative treatment to a drug therapy administration. As an example, the customized therapy may include sound therapy with one or more patient curated playlists. In one implementation, generating the customized therapy includes generating a non-drug therapeutic index for the patient. The customized therapy may be further based on one or more historical levels for the patient experience factor. The customized therapy may be combined with the drug therapy administration for an integrated treatment customized for the patient or may be an alternative to and result in an elimination of drug therapy.

An operation 312 generates an administration of a patient treatment experience based on the customized therapy. In one implementation, the patient treatment experience is generated using the at least one patient experience device and including one or more of patient sense stimulation and patient cognitive stimulation. The sense stimulation may include at least one of sight stimulation, taste stimulation, hearing stimulation, touch stimulation, or smell stimulation.

Biometric response data may be captured using at least one patient monitoring device, with the biometric response data captured during the administration of the patient experience treatment. An effectiveness of the customized therapy in addressing the current level of the patient experience factor may be determined based on the biometric response data. The patient experience treatment may be adjusted based on the effectiveness of the customized therapy. The patient experience treatment may be adjusted automatically. In one implementation, the patient experience treatment is adjusted until the effectiveness includes a target change in the patient experience factor. For example, the patient experience treatment includes playing at least one song, with a beats per minute of the at least one song being automatically adjusted based on the biometric response data of the patient until the target change is reached. The target change may include reaching a target biometric value. For example, the biometric response data includes a heart rate of the patient and the target biometric value includes a target heart rate, with the beats per minute of the at least one song being adjusted until the heart rate of the patient meets the target heart rate. A ranking of the effectiveness of the customized therapy among a plurality of customized therapies may be generated for the patient experience factor, with the ranking utilized to generate a patient management program for the patient. The patient management program may include an aggregated set of the plurality of customized therapies.

In one implementation, processed therapy data is generated by ingesting one or more of the biometric response data, the customized therapy, the effectiveness of the customized therapy, the patient experience data, and/or patient drug data. A baseline is generated for the patient experience factor and a patient profile is generated for the patient using the processed therapy data. A demographic profile may be generated based on the patient profile, and a demographic therapy for the demographic profile may be generated using the baseline for the patient experience factor. The demographic therapy may be selected for a second patient from a plurality of demographic therapies based on a match between the demographic profile with a second patient profile generated for the second patient. The demographic therapy may be customized for the second patient.

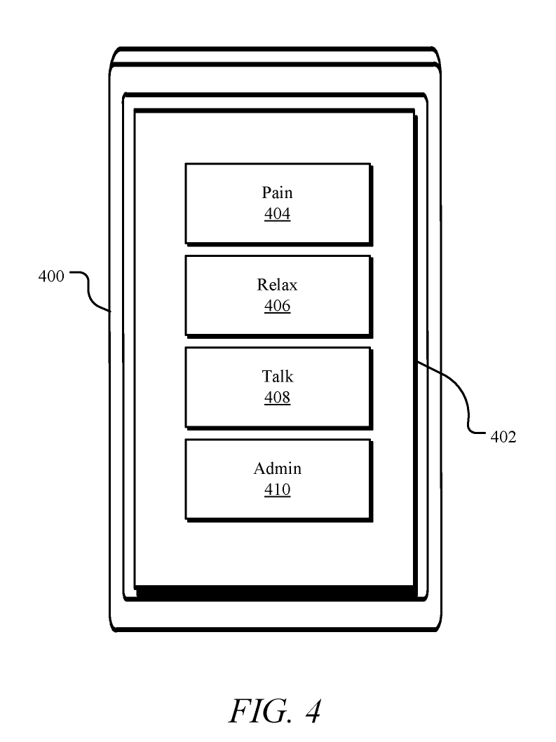

FIGS. 4-14 show example user interfaces generated by a computing device, such as one or more of the systems 102-110 and/or one or more of the components 202-212 of the patient experience components 200 of the treatment experience system 100, and displayed in a window of a user device 400 or other interface through which access to and interactions with the treatment experience systems and methods described herein and related data are provided. It will be appreciated by those skilled in the art that such depictions are exemplary only and not intended to be limiting.

Turning first to FIG. 4, an example experience user interface 402 generated by the treatment experience system 100. The experience user interface 402 may include one or more options to navigate to treatment experience resources and pages. For example, the experience user interface 402 may include a pain button 404, a relax button 406, a talk button 408, and an admin button 410. The pain button 404 provides resources for administering drugs, logging pain, and/or other patient experience factor data. The relax button 406 may be used to access customized therapies and/or to administer a corresponding patient experience treatment. The talk button 408 may connect the patient with a care professional, such as a nurse or other provider or caregiver, and/or make a call or send a message. The admin button 410 provides access to administrative resources for the treatment experience system 100.

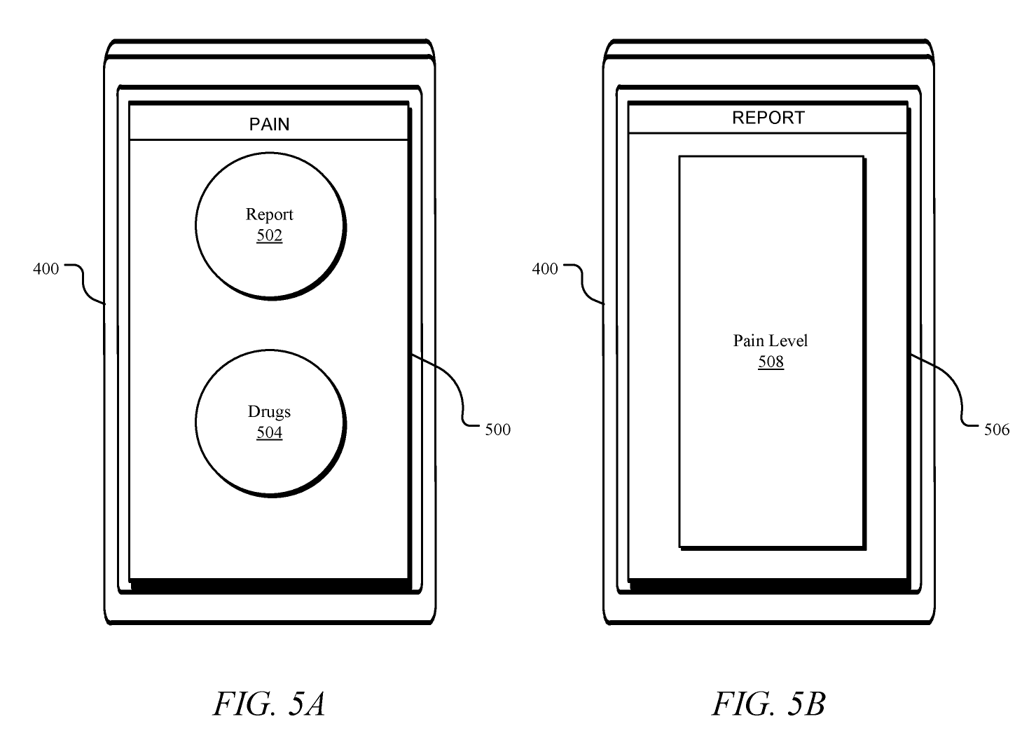

FIG. 5A depicts an example pain user interface 500 generated by the treatment experience system 100. In one implementation, the pain user interface 500 includes a report button 502 for accessing the experience factor assessment system 102 to capture patient experience data. For example, the report button 502 may navigate to an interface for logging pain. The drugs button 504 may be used to access the drug administration system 104 for information about or control of drug administration.

FIG. 5B shows an example pain reporting user interface 506 generated by the treatment experience system 100. In one implementation, the pain reporting user interface 506 is accessed via the report button 502. A pain level 508 interactive interface may be presented, for example, in the form of a scale or ladder, to report a current pain level, such as on a numerical scale between 0 and 10.

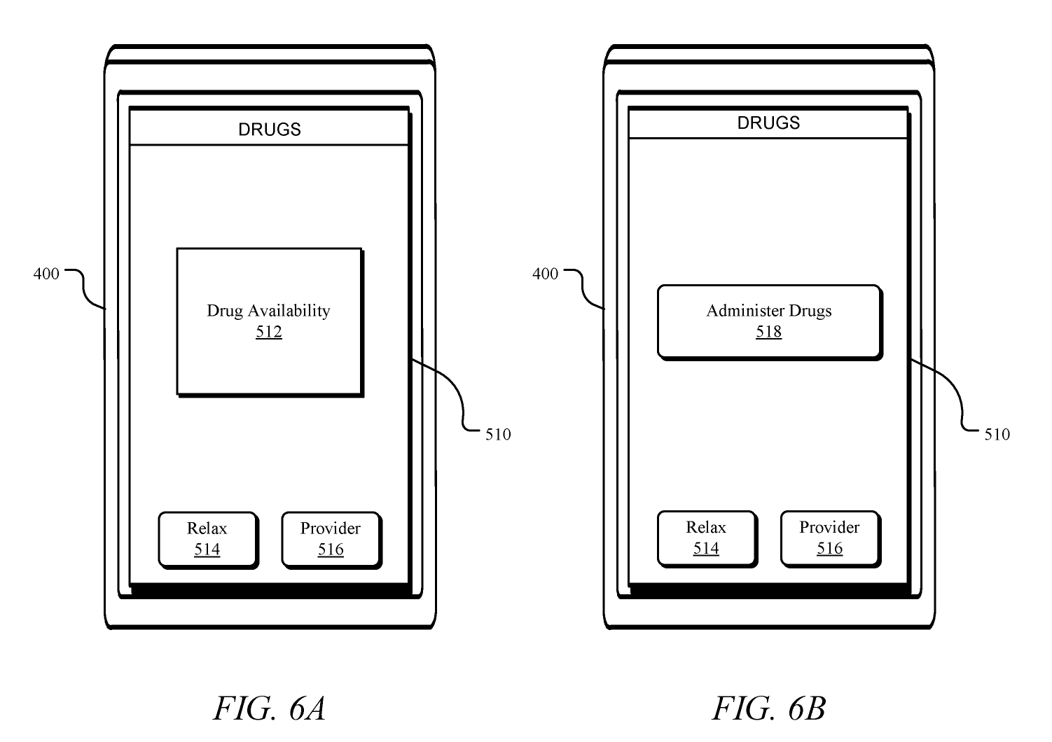

FIGS. 6A and 6B illustrate an example drug user interface 510 generated by the treatment experience system 100. In one implementation, the drug user interface 510 is accessed via the drugs button 504. After determining the current drug administration level for the patient, the drug user interface 510 either displays a drug availability interface 512 informing the patient of a time remaining until drugs may be taken or an administer drugs option 518 indicating to the patient that drugs may be administered if desired. The administer drugs option 512 may include a button for initiating the administration and/or send a message to a care provider to initiate the administration. To encourage the use of non-drug therapy, the drug user interface 510 may include a relax option 514 for accessing the customized therapy for the patient and initiating a patient treatment experience. A provider option 516 for contacting the care provider may also be presented.

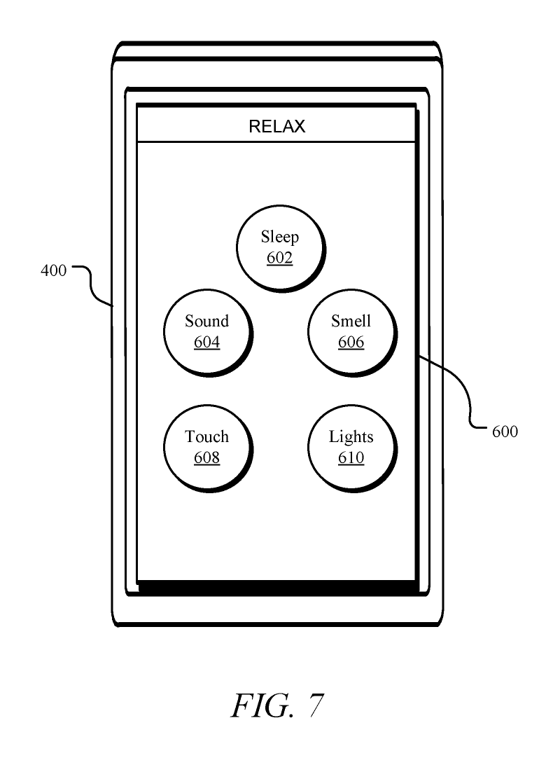

FIG. 7 shows an example relax user interface 700 generated by the treatment experience system 100. In one implementation, the relax user interface 700 includes various options for administering a patient treatment experience according to the customized therapy generated for the patient. The patient treatment experience may include one or more therapies directed at sense stimulation and/or cognitive stimulation. For example, the patient treatment experience may include options for sleep therapy 602, sound therapy 604, smell therapy 606, touch therapy 608, and light therapy 610.

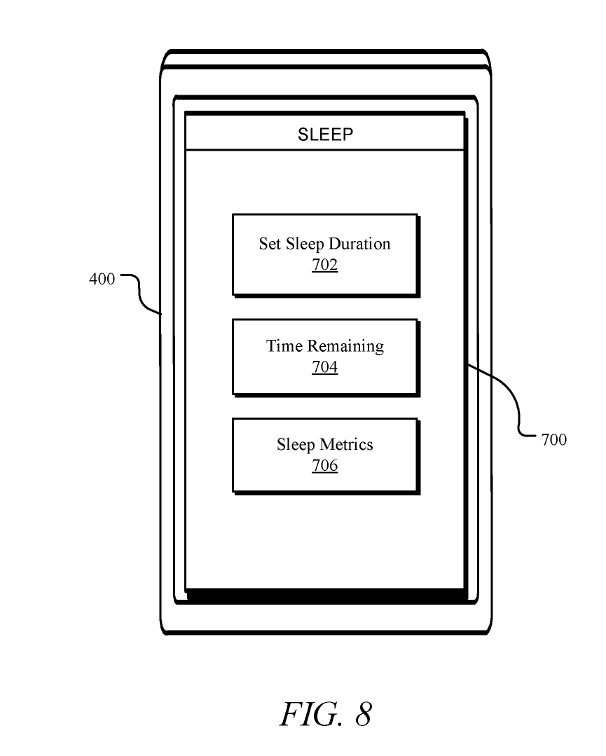

FIG. 8 depicts an example sleep therapy user interface 700 generated by the treatment experience system 100 and generated in response to selecting the sleep therapy option 602. In one implementation, the sleep therapy user interface 700 includes a set sleep duration option 702 to specify the amount of time to sleep, a time remaining display 704 providing information regarding remaining duration of the current sleep therapy session; and sleep metrics 706 for current and/or historical sleep therapy sessions.

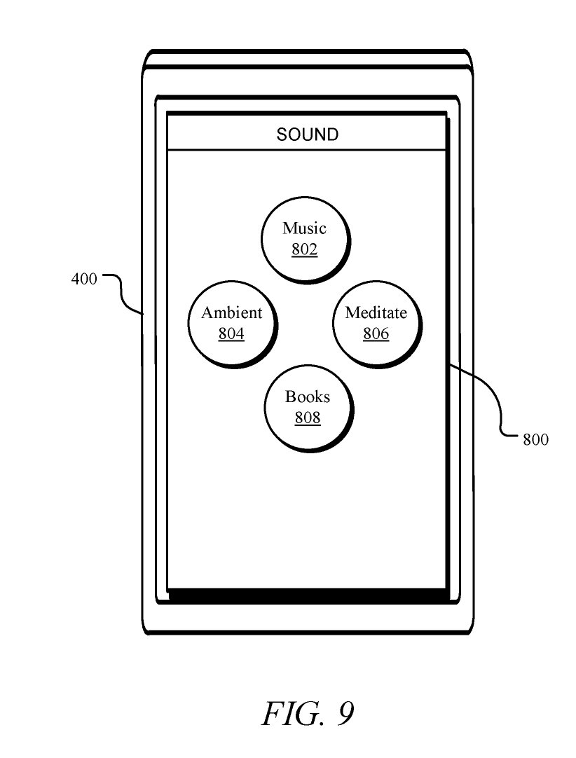

FIG. 9 depicts an example sound therapy user interface 800 generated by the treatment experience system 100 and generated in response to selecting the sound therapy option 604. In one implementation, the sound therapy user interface 800 includes various sound therapy options for the patient treatment experience, such as a music therapy option 802, an ambient noise therapy option 804, a meditate option 806, and a books option 808. The music therapy option 802 may give access to various music sources, and/or playlists that are automatically generated based on the customized therapy, patient curated, and updated in response to biometric response data. The ambient noise therapy option 804 may provide options for various types of ambient noise to listen to, such as white noise, campfire, rain, nature, oceans, heartbeats, etc. and may similarly include a playlist automatically generated based on the customized therapy, patient curated, and updated in response to biometric response data. The meditate option 806 may provide guided meditations for the patient to follow, including breathing and cognitive exercises, movements, and other meditative activities. The books option 808 may provide access to options for audio and/or visual books for listening or reading.

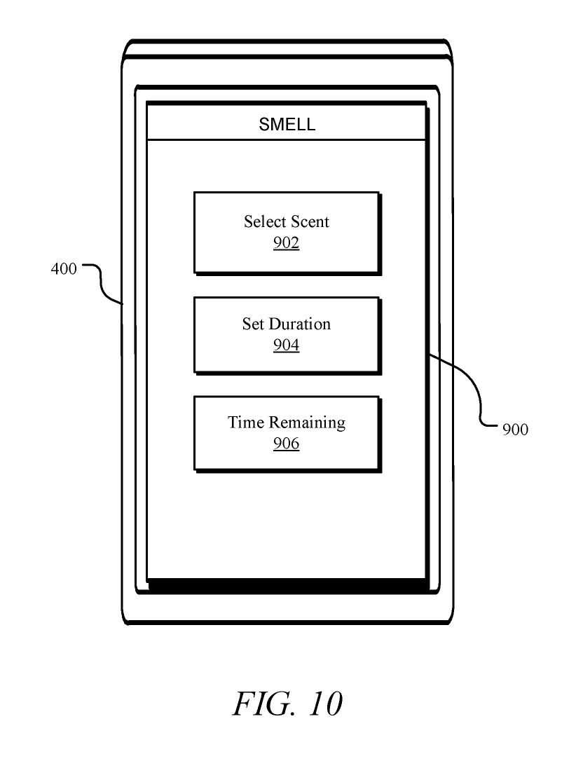

FIG. 10 shows an example smell therapy user interface 900 generated by the treatment experience system 100 and generated in response to selecting the smell therapy option 606. In one implementation, the smell therapy user interface 900 includes a select scent option 902 to select a scent type, such as lavender, cinnamon, mint, vanilla, and/or other scents selected for the patient based on the customized therapy. A set duration option 904 is presented to set the duration and interval of the scent. In one implementation, the smell therapy user interface 900 is used to control scents that are administered through a patient oxygen supply at the selected intervals and durations. A time remaining box 906 may provide information on time remaining during a current smell therapy session or time remaining until another session may be initiated.

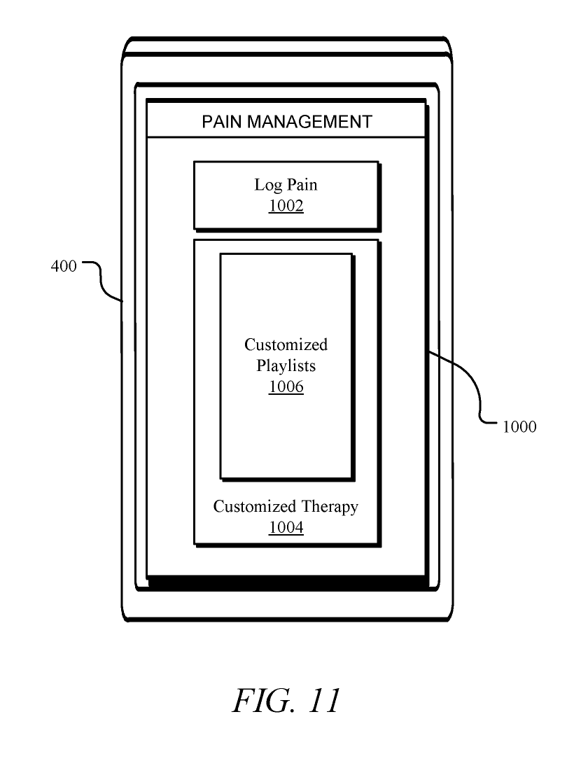

FIG. 11 illustrates an example pain management user interface 1000 generated by the treatment experience system 100. In one implementation, the pain management user interface 1000 includes a log pain option 1002 for reporting pain levels, a customized therapy 1004 with options for administering a patient treatment experience, such as customized playlists 1006 in connection with a sound therapy.

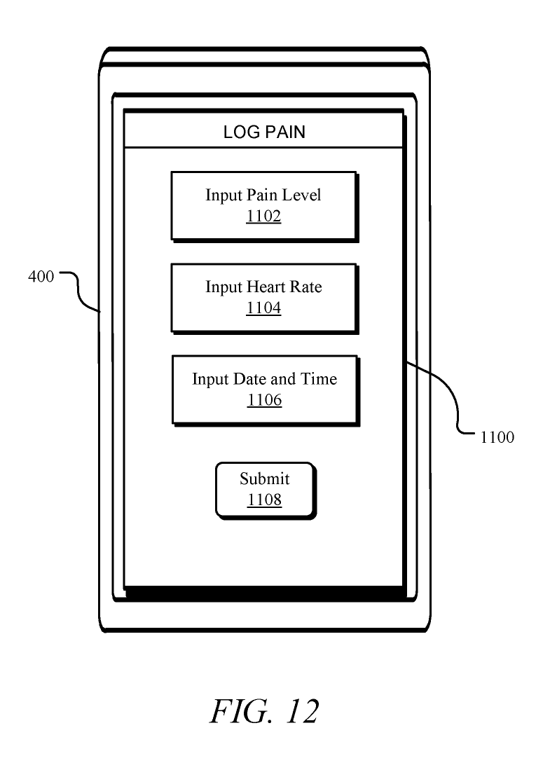

FIG. 12 depicts an example pain submission user interface 1100 generated by the treatment experience system 100. In one implementation, the pain submission user interface 1100 includes a pain level input 1102 indicating the current pain level input or otherwise determined for the patient, as well as options for adjusting the pain level; a heart rate input 1104 indicating the current heart rate for the patient, which may be modified; a date and time input 1106 detailing the current date and time associated with the pain level input 1102 and the heart rate input 1104; and a submit button 1108 for submitting the inputs 1102-1106. The inputs 1102-1106 may form part of the patient experience data, the current level of the patient experience factor (in this example pain), biometric response data, and/or target changes for biometric values (e.g., a target heat rate).

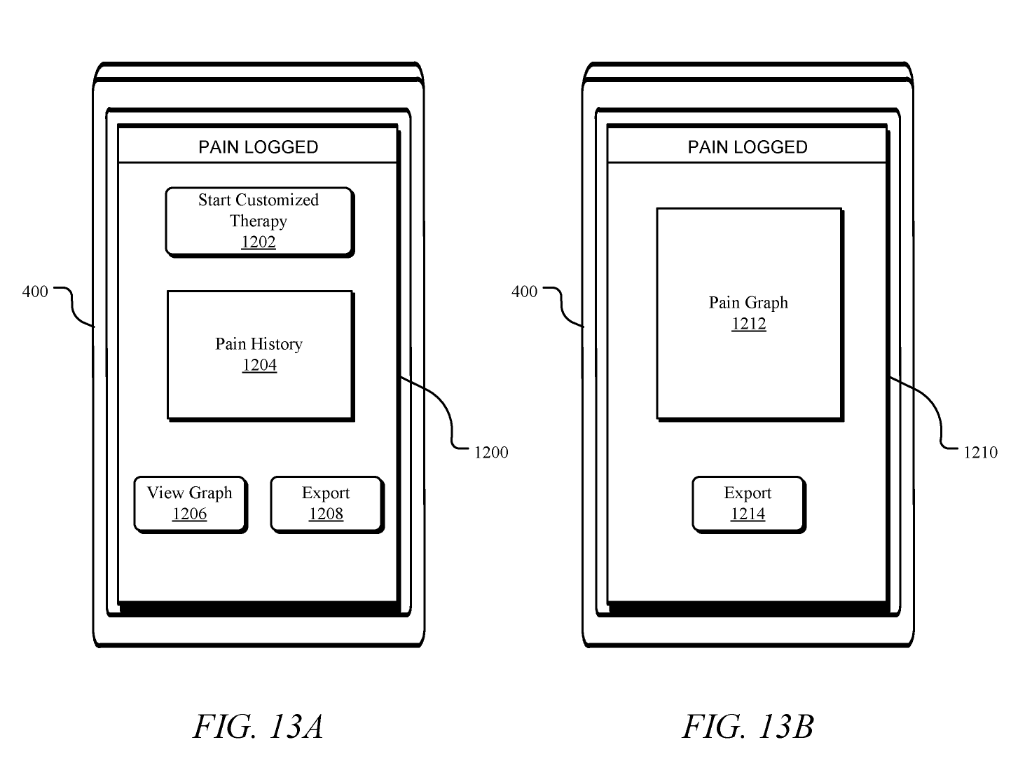

FIGS. 13A and 13B show example pain log user interfaces 1200 and 1210 generated by the treatment experience system 100. In one implementation, the pain log user interface 1200 includes an option to start customized therapy 1202. For example, the option 1202 may include a button to listen to music therapy. The pain log user interface 1200 may further include a pain history 1204, an option 1206 to view a pain graph 1212 of the pain log user interface 1210. Export options 1208 and 1214 may be presented to export processed therapy data, customized therapies, patient treatment experience therapies (e.g., playlists), biometric response data, patient experience data, and/or drug administration data. For example, the export buttons 1208 and 1214 may be used to export the pain history 1204 and/or the pain graph 1212.

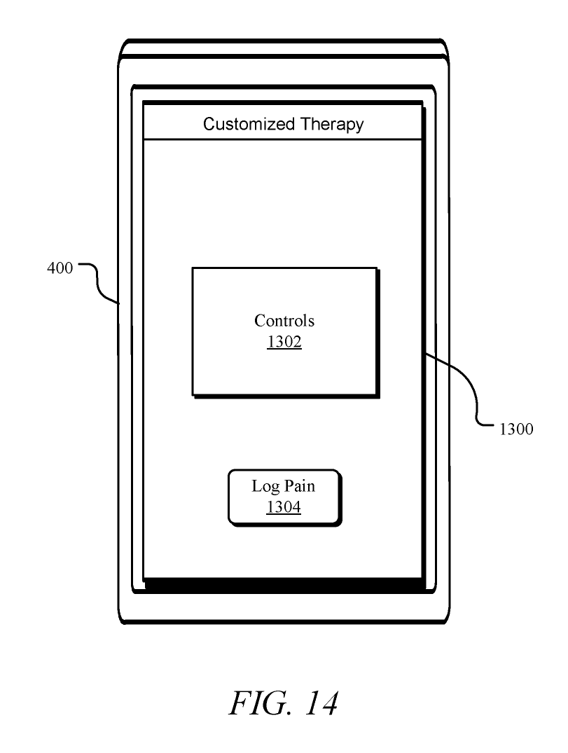

FIG. 14 depicts an example customized therapy user interface 1300 generated by the treatment experience system 100. In one implementation, the customized therapy user interface 1300 includes one or more controls 1302 for controlling the administration of the patient experience therapy, such as controls for navigating through one or more playlists. A log pain option 1304 directs the patient to the pain submission user interface 1100 to input a current pain level, which may be used to automatically update the administration of the patient experience therapy.

Other user interfaces may be provided for drug use data integration to measure an impact of the customized therapy on the patient experience factor(s). For example, with regard to chronic pain, a weekly survey of pain may be recorded on a chronic pain scale, such as the survey within a longitudinal case assessment. Patients will be assigned a regular series of pain management exercises to complete to help manage both chronic and acute episodes of Pain. Actual drug use for chronic pain patients will be correlated by accessing a comprehensive profile of patient drug use from a number of data sources, including, but not limited to: EHR [Electronic Health Record]; PDMP [Prescription Drug Monitoring Program]; toxicology data; lab data; patient prescription data; CDC drug use guidelines; and/or data from drug monitoring products. The assessments may ask the patient to rate their pain, enjoyment of life (and how pain has interfered with the same), and general activity (and how pain has interfered with the same) each week. The longitudinal case assessment may provide a management of chronic pain over years, where the patient reports a rating of whether pain is manageable, if the patient is feeling more or less anxious, if the patient is more or less active, whether the patient is increasingly forgetful, whether the patient has experienced any falls or other mobility issues, and whether the patient has been able to taper drug use.

Various example use cases are detailed below and are exemplary only and not intended to be limiting.

Use Case 0: Setup:

The treatment experience system 100 can accommodate either Setup in Advance Notice, such as Scheduled Surgery, Chronic Pain and/or Stress Recovery in a Home Environment, or No Advance Notice, such as Accident, Emergency Surgery, ICU and Trauma Ward Recovery in an Inpatient Facility setting. In either case, the steps for Setup will include, but not be limited to:

Determine Device Modality for Patient Acuity

Low Level—Patient Cannot See or Speak; Haptic Interface & Voice

Mid Level—Patient Can See and Speak briefly; Touch Screen & Voice

High Level—Patient Can See and Speak normally, Interacts with Basic Conversation; VR/AR Interface, Voice, Images, Video, Touch and/or Keypad

Enter Sociodemographic Baseline; Patient sociological and demographic data, such as age, sex, ethnicity, patient demographics, language, family history, medical history and Patient social media, music and personal experiences, or a sample set of sensory stimulation of smell, light, sound, touch, taste and thought for preferences as starting points to populate suggested customized therapy for discovery.

Enter Access Roles: Define who can access treatment experience system 100, what role they play in the Patient's interaction and recovery, such as Patient, Family Member, Nurse, Doctor, Facility, Administration, and the security/access rules that apply for each role.

Select Communication Templates: Preferences on How Patient will communicate with Family Members, Nurse, Doctor, Facility, Administration, Social Media and Extended Family. Questions from the Patient and Family are posted with Answers from the Nurse, Doctor or Facility that can be replayed and reviewed with the appropriate caregiver.

Select Learning Templates: Set Preferences for how Patient data representing Pain, Stress, Drugs and Alternative Therapies will be tracked and presented, such as graphs, text messages, voice alerts, and emails.

Select customized therapy templates: For each applicable Patient Alternative Therapy desired to be activated, if available, select source data for the treatment experience system 100 to access and scan for building a baseline template, including, but not limited to a Patient smartphone, smartwatch, wearable, hearable, lickable or computing device, social media accounts and other relevant personal information. For example, if the Patient has a music library or streaming account with individual playlists or preferences on a smartphone, wearable or computing device, that information would be accessed to discover Patient preferences to music genre, artists, songs and style for relaxation, meditation and/or comfort factors related to therapeutic healing.

Select Language: The Voice language option [Chinese, Spanish, English, Hindi, Arabic, Portuguese, Bengali, Russian, Japanese, Punjabi, German, French, Italian, etc.,] can be set and changed by Patient or User when the treatment experience system 100 is accessed

Select Preferences: If no Patient preference sources are available, the Administrator may be presented with a prebuilt or AI Machine generated series of information to build baseline templates for Patient Preferences of:
1. Sound; type, genre, playlists, styles, passage, pitch, modulation, frequency, metadata, volume, wavelength, duration
2. Sight; type, colors, wavelengths, modulation, intensity, duration, metadata
3. Smell; type, intensity, metadata, duration
4. Touch; type, intensity, metadata, duration
5. Taste; type, intensity, metadata, duration
6. Thought; type, intensity, metadata, duration

Select Drug Device and compound baseline: if available, the treatment experience system 100 will read and populate the available drug information, such as the drug type, dose and compliance information from the EMR, HIS and/or medical devices. If Drug Information is unavailable, and if applicable, manual entry of the baseline template will be provided.

Once the treatment experience system 100 setup has been completed, the various use cases that follow can be experienced. Based on the treatment experience system 100 modality and individual patient preferences, the patient will discover the most clinically appropriate complimentary medicine for alternative therapy to increase patient knowledge, manage pain, anxiety, stress, mobility and drug use while promoting a healing, therapeutic environment that improves the patient experience by monitoring and answering the following questions for each patient:

How does patient knowledge correlate with: Pain Levels? Stress Levels? Drug Use? Patient Experience?

How does alternative therapy correlate with: Pain Levels? Stress Levels? Drug Use? Patient Experience?

What other types of alternative therapies correlate with: Pain Levels? Stress Levels? Drug Use? Patient Experience?

What inter-relationships can be discovered and shared with others between these factors that enhance the patient experience?

Use Case 1:

Low Level Acuity: Voice User Interface [VUI] with Virtual AI Assistant

Trauma Patients are often confused, over stimulated, stressed, in pain, partially sedated and not operating at full cognitive capacity. In Use Case 1, the treatment experience system 100 input is binary and as simple as possible; as easy to use as possible, and as calm and therapeutic as possible. The input, such as a clicker button controller or haptic gesture controller could also function as a Bluetooth or 5G wireless technology trigger relay to initiate and operate the drug infusion pump.

The input controller and the treatment experience system 100 can be completely controlled with eyes shut, with a single push button or gesture. The device functionality could also be a voice driven assistant that would lead the Patient to actions based on simple Yes/No answers, indicated by the binary input. The Voice language option [Chinese, Spanish, English, Hindi, Arabic, Portuguese, Bengali, Russian, Japanese, Punjabi, German, French, Italian, etc.,] can be set and changed by patient or user when the treatment experience system 100 is accessed.

Modality:

Low Level—Patient cannot see or speak—click button/haptic interface & voice;

Mid-Level—Patient can see and speak briefly—touch screen & voice

High Level—Patient can see and speak normally, interacts with basic conversation—VA/AR interface, voice, images, video, touch and/or keypad

Patient input controller modality is set to Low Level—a simple button click or binary input, such as gesture, haptic signal on input device for yes/no interaction with generated VA voice application. Note: user interface joystick clicker button could be Yes/No, or a binary response haptic gesture detected by user interface motion sensor.

Setting: Headphones on, noise cancelling the machine alarms of the room. Patient's eyes are shut, avoiding the harsh lights, trying to get some rest. Patient does not want to speak, but she begins to experience increased pain.

Patient: Clicks button once to Awaken her Virtual Nurse [Voice Driven]

VN [on Headphones]: Hello Susan, are you in Pain? Please Click after the word to respond. Yes? [Pause indicates No. No Triggers Different Decision Tree . . . ]

Patient: Click [indicating Yes]

VN: Tell me your pain level on a scale of 1 to 10. I'll count and you click when we reach your Pain level. 1, 2, 3, 4, 5 . . .

Patient: Click [indicating Yes]

VN: Your Pain level is 5, is that right?

Patient: Click [indicating Yes]

[Stress Level calculated from biometric Vital Signs at Level 4]

VN: Do you need Pain Medication?

Patient: Click [indicating Yes]

VN: Ok, I see you have requested it; it should be arriving very soon.

Sound Therapy Option:

VN: Would you like me to play some music?

Patient: Click [indicating Yes]

VN: I'll Play your “Calm Playlist Now”. [Deeper Decision Tree for Type of Music]

Music plays . . .

Touch Therapy Option:

VN: Susan, while we wait, would you like to play a game?

Patient: Click [indicating Yes]

VN: I'll touch one of your fingers, and you touch back with that finger to respond.

The treatment experience system 100: Haptic signal to pinky.

Patient: Wiggles pinky.

VN: Great! I feel you. Now I'll give you a sequence to repeat back.

The treatment experience system 100: Haptic signal to pinky, then thumb, then middle finger.

Patient: Repeats haptic pattern.

VN: You are doing fantastic! How about a hand massage?

Patient: Yes

The treatment experience system 100: Vibrate all sensors at once.

VN: Touch your hand where you would like stimulation. Does that feel good?

Patient: Yes

Other Alternative Therapy Options:

[Sight Therapy]

[Smell Therapy]

[Thought Therapy—Behavioral Health]

[Taste Therapy]

VN: You are doing great, Susan. Do you need anything else?

Patient: No Response [Indicates No]

VN: I'll let you get some rest. Just ‘Call’ [gesture, button] me if you need me . . .

Or

VN: Would you like me to let you know when more pain medication can be accessed?

Patient: Click yes

VN: Ok; When more Pain Medication is available, I′ll play let you know. [Calm chord, haptic vibration and green light] If you need your available Pain Medication when the Sound Plays, feel free to request it. Here's a thought—try listening to music for a little while instead of starting your pain medications immediately. Of course, any time you feel you need the pain medications, they are available at your request; you can decide.

VN: Chord plays

VN: Medications arriving in 15 seconds, 14, 13, 12 . . . medications available now.

[Patient initiates Drug Infusion]

If Vital Signs show increased Stress Level per parameters—

VN: Susan, do you need me to call your nurse?

Patient: Click Yes

VN: I have called Sharon [your nurse]; she will be her in a moment. We will address your issues and try to make you more comfortable . . .

The Virtual Nurse becomes the digital Patient Assistant for the low level, non-emergency interaction, freeing the Nurse for critical Care.

Nurse at Station with VN

Nurse: Hello PEG. Can I have your Patient Report for room 4118?

VN: Patient 4118 is Susan Jones. Susan woke me at 10:30 pm to report that her Pain Level had increased from 3 to 5. Stress Level was calculated at level 4. Based on your Alert Request Protocol, here is my Report [display or Voice]:

Reporting Log:

Display [or Voice]

Patient Report for Susan Jones, Room 4118

### Sep. 17, 20XX

| | |
|---|---|
| 10:05:30 PM Pain Level 5 | [Patient Reported] |
| 10:06:30 PM Drug Access | [Patient Requested] |
| 10:06:31 PM Vitals Verified: | [Details] |
| Within Tolerance | |
| 10:06:33 PM Drug Infusion Initiated | [Patient Initiated]; |
| | 10 ml/10 min cycle |
| 10:06:33 PM Stress Level 4 | [Calculated] |
| 10:06:45 PM Music | [Patient Requested: Marconi] |
| 10:16:33 PM Pain Level 3 | [Patient Reported] |
| 10:16:33 PM Vitals Verified: Lowered | [Details] |
| 10:17:03 PM Drug Access | [No Patient Request] |
| 10:17:03 PM Stress Level 3 | [Calculated] |
| 10:30:00 PM Do Not Disturb | [Patient Requested] |
| 10:35:00 PM Sleep Detected: Deep | [Biometric Details] |

Nurse: Report Noted. Have you Learned anything I should be aware of?

VN: Yes, Susan's Stress Level, Pain Levels and key Vital Signs are decreasing without additional Drugs Requested—I have Learned that her response to Music with the Song Weightless by Marconi Union is positive. Susan has requested Do Not Disturb, so I will not wake her for Pain Med Availability unless she calls.

Nurse: Thank you PEG. That is all—GOOD BYE.

VN: You are welcome. I've updated your treatment log and will Alert you if Patient 4118 needs you . . . GOOD BYE.

Use Case 2:

Medium Level Acuity: Touch Screen Interface [GUI] Sample Use Case

Modalities:

Low Level—Patient Cannot See or Speak—Haptic Interface & Voice

Medium Level—Patient Can See and Speak briefly—Touch Screen & Voice

High Level—Patient Can See and Speak normally, Interacts with Basic Conversation—VR/AR Interface, Voice, Images, Video, Touch and/or Keypad

Patient Input Controller Modality is set to Medium Level—Voice driven interface [VUI] with additional Touch Screen and/or Haptic inputs [HUI]. A simple Button Click, such as Haptic gesture, Button Click or Touch Screen [GUI] response on the input device for Yes/No Interaction with Generated VA Voice Application. Note: User may alternate between User Interface modalities depending on responsiveness.

Setting: The Patient headphones are on/off [depending on Patient alertness] for optional noise cancelling the medical device alarms in the room. Patient's eyes are open for short periods of time, avoiding the harsh lights, trying to get some rest. Patient can speak briefly but closes eyes when she begins to experience increased pain.

Patient: Clicks button, touches screen or verbally calls to Awaken her Virtual Nurse [Voice Driven]

VN [on Headphones]: Hello Susan, are you in Pain? Please Say YES [or Click, Touch]after the word to respond. Yes? [Pause indicates No. No Triggers Different Decision Tree . . . ]

Patient: Verbal Yes [indicating Yes]

VN: Tell me your pain level on a scale of 1 to 10. I'll count and you click when we reach your Pain level. 1, 2, 3, 4, 5 . . .

Patient: Yes

VN: Your Pain level is 5, is that right?

Patient: Yes

[Stress Level calculated from biometric Vital Signs at Level 4]

VN: Do you need Pain Medication?

Patient: Yes

VN: Ok, I see you have requested it; it should be arriving very soon.

Sound Therapy Option:

VN: Would you like me to play some music?

Patient: Yes

VN: I'll Play your “Calm Playlist Now”. [Deeper Decision Tree for Type of Music]

Music plays . . .

Touch Therapy Option:

VN: Susan, while we wait, would you like to play a game?

Patient: Yes

VN: I'll touch one of your fingers, and you touch back with that finger to respond.

The treatment experience system 100: Haptic signal to pinky.

Patient: Wiggles pinky.

VN: Great! I feel you. Now I'll give you a sequence to repeat back.

The treatment experience system 100: Haptic signal to pinky, then thumb, then middle finger.

Patient: Repeats haptic pattern.

VN: You are doing fantastic! Would you like a hand massage?

Patient: Yes

The treatment experience system 100: Vibrates all sensors at once.

VN: Susan, touch your hand where you would like stimulation. Does that feel good?

Patient: Yes

Social Interaction Option:

VN: Susan, I see you have some messages, would you like to listen to them?

Patient: Yes

VN: Here's your messages—Say Yes when I identify a message to play—Mom . . . Amanda . . . Grandmother . . .

Patient: Yes

VN: You have a message from Grandmother . . . Play Message—“Hi peanut, I just wanted you to know we are all thinking of you. You just rest and get better—we will see you soon! We love you!”

VN: Would you like to leave a response for Grandmother?

Patient: Yes

VN: Leave your message after the chord . . . Chord Plays.

Patient: Hi Grandma, thanks for thinking of me . . . I love you.

VN: I'll post you message to Grandmother.

Additional Alternative Therapy Options:

[Taste Therapy]

[Sight Therapy]

[Smell Therapy]

[Thought Therapy]

VN: You are doing great, Susan. Do you need anything else?

Patient: No

VN: I'll let you get some rest. Just ‘Call’ [Say Call, gesture, button, touch] me if you need me . . .

Or

VN: Would you like me to let you know when more Pain Medication can be accessed? Display [green light, chord]?

Patient: Yes

VN: Ok; When more Pain Medication is available, I'll play let you know. [Calm chord, haptic vibration and green light] If you need your available Pain Medication when the Sound Plays, feel free to request it. Here's a thought—try listening to music for a little while instead of starting your pain medications immediately. Of course, any time you feel you need the pain medications, they are available at your request; you can decide.

VN: Chord plays

VN: Medications arriving in 15 seconds, 14, 13, 12 . . . medications initiated now.

[Drug Infusion being delivered]

Or Vital Signs show increased Stress Level

VN: Susan, do you need me to call your nurse?

Patient: Yes

VN: I have called Sharon [your nurse]; she will be her in a moment. We will address your issues and try to make you more comfortable.

The Virtual Nurse becomes the digital Patient Assistant for the low level, non-emergency interaction, freeing the Nurse for critical Care.

Nurse at Station with VN

Nurse: Hello PEG. Can I have your Patient Report for room 4118?

VN: Patient 4118 is Susan Jones. Susan woke me at 10:30 pm to report that her Pain Level had increased from 3 to 5. Stress Level was calculated at level 4. Based on your Alert Request Protocol, here is my Report [display or Voice]:

Use Case 3:

High Level Acuity: Outpatient or Monitored Home Health Use; may be in a variety of care facilities, such as Independent Living Facility, Assisted Living, or Patient's private residence: Virtual/Augmented Reality Interface [VR/AR] Sample Use Case.

Modalities:

Low Level—Patient Cannot See or Speak—Haptic Interface & Voice

Medium Level—Patient Can See and Speak briefly—Touch Screen & Voice

High Level—Patient Can See and Speak normally, Interacts with Basic Conversation—VR/AR Interface, Voice, Images, Video, Touch and/or Keypad

Patient Input Controller Modality is set to HIGH Level—Voice driven interface [VUI] or GUI with additional inputs, such as a VR/AR headset, Holographic glasses and Haptic inputs [HUI]. The VR/AR or Holographic Interface may use a variety of input signals, including, but not limited to, head tracking, controllers, hand tracking, voice, Haptic signals, on-device buttons or trackpads. A simple Button Click, Haptic gesture or head movement is detected by the input device for Yes/No Interaction with the VA Voice or GUI Application. Note: User may alternate between User Interface modalities depending on responsiveness.

Setting: The Patient headset, glasses and/or headphones are set for noise cancelling, eliminating the unwanted noise in the room. Patient's eyes are open [within headset or glasses] for extended periods of time by Light Therapy that avoids the harsh lighting, and the Patient can actively engage the Virtual/Augmented Reality experience. Patient can speak but may close eyes and refocuses her mind body controls when she begins to experience increase pain.

Patient: Clicks button, touches screen or verbally calls to Awaken her Virtual Nurse [Voice Driven]

VN [on Headphones]: Hello Susan, are you in Pain? Please Say YES after my words to respond, or [Click, Touch, Gesture] Yes?

[Pause indicates No. ‘No’ Triggers Different VN VUI Decision Tree . . . ]

Patient: Head Gesture Yes [indicating Yes]

VN: Tell me your pain level on a scale of 1 to 10. I'll count and you click when we reach your Pain level. 1, 2, 3, 4, 5 . . .

Patient: Yes

VN: Your Pain level is 5, is that right?

Patient: Yes

[Stress Level calculated from biometric Vital Signs at Level 4]

VN: Do you need your Pain Medication?

Patient: Yes

VN: Ok, I see you have requested it; it should be arriving very soon.

Alternative Therapy Options May Be Presented to Patient

Light Therapy Option

VN: Would you like me to change the lighting?

Patient: Yes

VN: I'll turn off the fluorescent lights, and gradually add a softer hue. In the room, I'll also add some natural sunlight [to stimulate production of vitamin D and serotonin, making the Patient's mood happier].

VN: Do you still have a stiff neck?

Patient: Yes

VN: I'll activate the light emitting diodes in your neck pillow and a mild massage [Light Therapy to trigger natural intracellular photobiochemical reactions, and Haptic vibrations for massage]. Does that feel better?

Patient: Yes

VN: Good, I'll check back in 15 minutes to see if you pain level improves.

Sound Therapy Option:

VN: Would you like me to play some music?

Patient: Yes

VN: I'll Play your “Calm Playlist Now”. [Deeper Decision Tree for Type of Music]

Music plays . . .

Touch Therapy Option:

VN: Susan, while we wait, would you like to play a game?

Patient: Yes

VN: I'll touch one of your fingers, and you touch back with that finger to respond.

The treatment experience system 100: Haptic signal to pinky.

Patient: Wiggles pinky.

VN: Great! I feel you. Now I'll give you a sequence to repeat back.

The treatment experience system 100: Haptic signal to pinky, then thumb, then middle finger.

Patient: Repeats haptic pattern.

VN: You are doing fantastic! How about a hand massage?

Patient: Yes

The treatment experience system 100: Vibrate all sensors at once.

VN: Touch your hand where you would like stimulation. Does that feel good?

Patient: Yes

Social Interaction Option—Thought:

VN: Susan, I see you have some messages, would you like to listen to them?

Patient: Yes

VN: Here's your messages—Say Yes when I identify the message—Mom . . . Amanda . . . Grandmother . . .

Patient: Yes

VN: You have a message from Grandmother . . . Play Message—“Hi Susan, I just wanted you to know we are all thinking of you. You just rest and get better—we will see you soon! We love you!”

VN: Would you like to leave a response for Grandmother?

Patient: Yes

VN: Leave your message after the chord . . . Chord Plays.

Patient: Hi Grandma, thanks for thinking of me . . . I love you.

VN: I'll post you message to Grandmother.

AR/Smell Therapy Option:

VN: Susan, while we wait, would you like relax?

Patient: Yes

VN: I see you enjoy hikes in the mountains and the smell of flowers.

Patient: Yes

VN: I like mountain hikes too. I going to play some natural sounds and show you a video of the mountains. Try to relax your breathing and come with me. After a bit, you can close your eyes if you would like. Are you ready?

Patient: Yes

[Natural sounds play, a stream, wind and occasional birds chirping. The mountain hike video starts.]

VN: Susan, imagine that you in the mountains, taking a walk. I see a field of lavender up ahead—raise your glove to your nose—can you smell it?

[The treatment experience system Controller olfactory unit heats lavender oil in glove for relaxing scent diffusion. Patient inhales, smiles and exhales]

VN: Doses that smell good, Susan?

Patient: Nods Yes.

VN: You are doing great, Susan. Do you need anything else?

Patient: No

VN: I'll let you get some rest. Just ‘Call’ [Say Call, gesture, button, touch] me if you need me . . .

Or

VN: Would you like me to let you know when more Pain Medication can be accessed? [green light, chord]?

Patient: Yes

VN: Ok; When more Pain Medication is available, I'll play let you know. [Calm chord, haptic vibration and green light] If you need your available Pain Medication when the Sound Plays, feel free to request it. Here's a thought—try listening to music for a little while instead of starting your pain medications immediately. Of course, any time you feel you need the pain medications, they are available at your request; you can decide.

VN: Chord plays

VN: Medications arriving in 15 seconds, 14, 13, 12 . . . medications initiated now.

[Drug Infusion being delivered]

Or Vital Signs show increased Stress Level

VN: Susan, do you need me to call your nurse?

Patient: Yes

VN: I have called Sharon [your nurse]; she will be her in a moment. We will address your issues and try to make you more comfortable . . .

The Virtual Nurse becomes the digital Patient Assistant for the low level, non-emergency interaction, freeing the Nurse for critical Care.

Nurse at Station with VN

Nurse: Hello PEG. Can I have your Patient Report for room 4118?

VN: Patient 4118 is Susan Jones. Susan woke me at 10:30 pm to report that her Pain Level had increased from 3 to 5. Stress Level was calculated at level 4. Based on your Alert Request Protocol, I here is my Report [display or Voice].

Use Case 4: Home Setting

Under the Home Setting, no Patient Health Information is shared with any HIPPA providers. The App operates as a mindfulness and wellness App, not a medical device, and provides no medical advice. No Patient Health Information is collected, and if Patients are suffering from stress, anxiety, pain or mobility issues, they are directed to consult a doctor or healthcare professional.

As an option, Patients may select to share their pain experience tracking data by ‘Opting In’ within the treatment experience system 100 for therapist sharing with her medical provider or behavioral health therapist. In this optional use case, all HIPPA regulations are followed.

Virtual/Augmented Reality Interface [VR/AR] Sample Use Case

Modalities:

Low Level—Patient Cannot See or Speak—Haptic Interface & Voice

Medium Level—Patient Can See and Speak briefly—Touch Screen & Voice

High Level—Patient Can See and Speak normally, Interacts with Basic Conversation—VR/AR Interface, Voice, Images, Video, Touch and/or Keypad

Patient Input Controller Modality is set to HIGH Level—Voice driven interface [VUI] or GUI with additional inputs, such as a VR/AR headset, Holographic glasses and Haptic inputs [HUI]. The VR/AR or Holographic Interface may use a variety of input signals, including, but not limited to, head tracking, controllers, hand tracking, voice, Haptic signals, on-device buttons or trackpads. A simple Button Click, Haptic gesture or head movement is detected by the input device for Yes/No Interaction with the VA Voice or GUI Application. Note: User may alternate between User Interface modalities depending on responsiveness.

Setting: The Patient headset, glasses and/or headphones are set for noise cancellation, eliminating unwanted noise in the room. Patient's eyes are open [within headset or glasses] for extended periods of time, allowing use of Light Therapy that avoids the harsh lighting, and the Patient can actively engage the Virtual/Augmented Reality experience. Patient can speak freely but may close eyes and refocus her mind/body controls when she begins to experience episodic pain.

App Setup: Building a baseline profile will help the app learn about you, and how you can begin to influence and manage pain, stress and anxiety with music, tailored to your needs.

Pain Log: Recording a weekly Pain Log will help you establish a baseline to understand and assess your progress.

Alternative Therapy Preferences [Music]:

To help identify the types of music that you find relaxing, listen to and rate a sample soundscape from each Genre on the Preferences page. Once you start using your music selections to manage pain, stress and anxiety, you will start to learn how a soundscape or song affects your mind and body.

Episodes [Factor could be Pain, Anxiety, Stress, Mobility]

Establish a quiet moment at least once a day, or whenever you feel an episode of pain, stress, anxiety or mobility to record your vital signs and then listen to a selected soundscape or song. On the Listen page, you can access your Preferred music.

Select a Music Genre & Log Your Vitals

Find a Quiet Place, Sit or Recline

Use Headphones if Available

Center Yourself, Close Your Eyes, Breath Slowly and Listen

After Listening, Log Your Vitals & Verify Your Progress

Diary [Factor of Pain, Stress, Anxiety or Mobility]

If you experience a Pain Episode, after it is over, take a moment to enter how it felt in your Pain Diary. This will help you and your doctor understand the causes of your pain and formulate the most effective treatment for you.

Dashboard of Progress

Here you will find a “Dashboard” and Insights of your current progress and how you are managing your pain, stress, anxiety and mobility issues.

Sample Dashboard Views

Baseline Profile

Preferences

Pain Log & history [Chronic]

Pain Episodes & history [Acute]

Music Applications Used by Genre, Song

Music Effectiveness by Genre, Song

Music Impact on Pain [+/−Pain Effectiveness]

Training Exercises

A collection of the best licensed content and self-guided exercise programs for enhancing your relaxation experience. Your Pain [Anxiety, Stress, Mobility] is not solely managed by relaxation, sometimes you may want to ‘burn off’ anxious feelings or energy in an exercise routine; or you may chose a slower, more deliberate low impact workout. The Pages Allegro[fast] and Legassimo [slow] provide some music for just those occasions.

Sleep:

When you want to unwind or no without a log; no tracking pain, heart rate or respiration—just breathe. Experience some relaxing music and soundscapes selected specifically for you by the treatment experience system 100 for your sleep or mindful meditation.

Share

If you choose to ‘Opt In’, the treatment experience system 100 will share your tracking data with your approved therapist or medical provider.

Keeping a weekly Pain Log can help you and your doctor assess your progress, and the effectiveness of your treatment plan. Pick one day a week to update your Pain Log. Pain doctors also recommend keeping a Pain Diary to establish a consistent record of your pain episodes and experience. This can become a method to help your doctor better assess your treatment plan and progress. When she asks, ‘how have you have been in the past month or two’, you can provide helpful specifics.

Patient: Initiates the treatment experience system 100 or Clicks button, touches screen or verbally calls to Awaken her Virtual Nurse [Voice Driven]

VN [on Headphones or App Text]: Hello Susan, are you in Pain? Please Say YES after my words to respond, or [Click, Touch, Gesture] Yes?

[Pause indicates No. ‘No’ Triggers Different VN VUI Decision Tree . . . ]

Patient: Head Gesture Yes [indicating Yes]

VN: Select your Pain Level on a scale of 1 to 10, then either tell me your Heart Rate and Respiration Rate, or I will read them from your wearable monitor.

Patient: Selects Pain; the treatment experience system 100 reads Heart Rate and Respiration Rate from wearable API.

VN: Your Pain level is 5, is that right?

Patient: Yes

[Stress Level calculated from biometric Vital Signs at Level 4]

VN: Ok, you are strong. You know how to handle this pain, you have done it before.

Let's try listening to music for a little while. If you are under the care of a doctor, please follow her directions for use of any pain medications.

VN: Playing you ‘Calm Playlist’ now.

Based on Individual Machine Learning—other Alternative Therapy Options May Be Presented to Patient

Light Therapy Option:

VN: The lights are pretty bright, would you like me to soften the lighting?

Patient: Yes

VN: I'll turn off the fluorescent lights [IoT lighting interface] and gradually add a softer hue. I'll also add some natural sunlight [to stimulate production of vitamin D and serotonin, making the Patient's mood happier].

VN: Do you still have a stiff neck?

Patient: Yes

VN: I'll activate the light emitting diodes in your neck pillow and a mild massage [Light Therapy to trigger natural intracellular photobiochemical reactions, and Haptic vibrations for massage].

VN: Does that feel better?

Patient: Yes

VN: Good, I'll check back in 15 minutes to see if you pain level improves.

Sound Therapy Option:

VN: Would you like me to play some more music?

Patient: Yes

VN: I'll Play your ‘Sleep Playlist’ now.

[Deeper Decision Tree for Type of Music]

Music plays . . .

The treatment experience system 100 monitors wearable heart rate and respiration for episode; they are improving.

Touch Therapy Option—provide distraction

VN: Susan, you are doing great. Would you like to play a game?

Patient: Yes

VN: I'll touch one of your fingers, and you touch back with that finger to respond.

The treatment experience system 100: Haptic signal to pinky.

Patient: Wiggles pinky.

VN: Great! I feel you. Now I'll give you a sequence to repeat back.

The treatment experience system 100: Haptic signal to pinky, then thumb, then middle finger.

Patient: Repeats haptic pattern.

VN: You are doing fantastic! How about a hand massage?

Patient: Yes

The treatment experience system 100: Vibrate all sensors at once.

VN: Touch your hand where you would like stimulation. Does that feel good?

Patient: Yes

AR/Smell Therapy Option

VN: Susan, while we wait, would you like relax?

Patient: Yes

VN: I see you enjoy hikes in the mountains and the smell of flowers.

Patient: Yes

VN: I like mountain hikes too. I going to play some natural sounds and show you a video of the mountains. Try to relax your breathing and come with me. After a bit, you can close your eyes if you would like. Are you ready?

Patient: Yes

[Natural sounds play, a stream, wind and occasional birds chirping. The mountain hike video starts.]

VN: Susan, imagine that you in the mountains, taking a walk. I see a field of lavender up ahead—raise your glove to your nose—can you smell it?

[The treatment experience system 100 controller olfactory unit heats lavender oil in glove for relaxing scent diffusion. Patient inhales, smiles and exhales]

VN: Doses that smell good, Susan?

Patient: Nods Yes.

VN: You are doing great, Susan; you've got this under control. Let's recheck you Pain level and vital signs.

VN: Select your Pain Level on a scale of 1 to 10.

Patient: Selects Pain; the treatment experience system 100 reads Heart Rate and Respiration Rate from wearable API.

VN: Your Pain level is 2, is that right?

Patient: Yes

[Stress Level calculated from biometric Vital Signs at Level 2]

VN: Ok, you are strong. Pain does not own you; you own pain. Try and relax for a while to the music. If you have ‘OPTED IN” for sharing your experience with your therapist or doctor, would you like me to share it now? She will be proud of the progress you are making.

In each Use Case, the Patient Experiences and Feedback are recorded for the history and effectiveness of using Alternative Therapy(s) to manage Pain, Stress, Anxiety and Mobility while Mitigating Drug Use. This information is presented in a summary Dashboard, with data drill downs, to the Patient, Family, Caregivers, Nurse and Doctors for determination of the most appropriate Cooperative Medicine treatment protocols to improve the Patient Experience while recovering from an injury, trauma or Chronic Pain situation. The treatment experience system 100 will also assist the Patient and Family in the most accurate and beneficial evaluation of the Caregivers and Facility in the pursuit of the highest quality, lowest cost healthcare experience for effective Pain and Stress mitigation programs that lower Drug Use.

Government Health Organizations, such as Medicare and Medicaid, are shifting financial reimbursement models from traditional payment mechanisms, such as Fee For Service, to a Population Health model, emphasizing Value Based Outcomes for providing the highest quality, low cost healthcare outcomes for individuals and at risk Patient groups. A key part of this reimbursement model shift is driven by an accurate and timely assessment of the Patient Experience in healthcare delivery.

The treatment experience system 100 contributes valuable, quantifiable insight to the Patient Experience Evaluation, sometimes represented by Survey Questions, as it reflects key areas of Patient Treatment and Clinical methods, highlighted in recent Medicare sponsored guideline Questions for assessing the Patient Experience [sample outlined below]:

The treatment experience system 100 is an intelligent Software Platform that discovers the Pain Effectiveness of Music for an individual Patient and then uses Metadata, Patient Behavior and Machine Learning/AI to discover and share Music for Pain Effectiveness for similar Patients.

The treatment experience system 100 is based on a cognitive behavioral model of therapy, which posits that new thoughts, feelings and body states may be conditioned to replace dysfunctional patterns. Specifically, a relaxed body and pleasant visual images may replace tension and worry when they are conditioned as a response mechanism with familiar, calming music. The therapeutic music discovery framework in the treatment experience system 100 is designed to perform several functions:
Direct attention away from pain, distracting the listener with comforting music
Provide a musical stimulus for targeted rhythmic breathing
Offer a rhythmic structure for systematic release of body tension
Cue positive visual imagery
Condition a deep relaxation response
Change mood
Focus on positive thoughts and feelings to celebrate life
Algorithmic Rules and Machine Learning/AI System and Methods
Familiarity
Build Playlists for Music Preference Probability for Familiarity with Machine Learning, using Survey, Social Media, Family
Age Band
Build Playlists for Music Preference for Age Band with Machine Learning, extracting and targeting Age/Sex/Demographic ranges, such as 12-22, 35-45 . . .
Metadata
Build Playlists for Music Preference for Metadata matches of
Genre/Origin/Era/Artist/Type/Tempo/Mood/BPM/Keywords
Source, Health Condition/Pain Type/Pain Effectiveness/Demographic Profile
Self-Chosen
Build Playlists for Music Preference for Self-Chosen
Genre/Mood/Artist/Song
Non-Music Sounds
Build Playlists for Music Preference for
Nature
Binaural Beats
Solfeggio Frequencies
Wavelength Vibrations
Biometric and Vital Signs
Build Playlists for Music Preference for impact of/feedback from Biometric and Vital Signs
Machine Curation
Build Playlists for Music Preference and Machine Learning for
Individual
Demographic
Projection
Machine Learning Engine
Music Discovery

Research studies have indicated that one of the best predictors for establishing successful therapeutic music in factor relief is to present patient selected music during a Factor episode. When accessing Sound and Music as the targeted Alternative Therapy, the treatment experience system 100 will attempt to establish a patient's musical preferences using one or more baseline techniques by presenting options for the patient to indicate a ‘like’ or ‘don't like’ response. The ‘like’ responses, and the metadata associated with the patient preferences, are then used to build initial playlists for the factor management. These preferences are ‘learned’ as a starting point by the treatment experience system 100 machine learning engine, using a variety of techniques.

For example the patient is asked to name or select several music or sound representations of what they feel are relaxing or soothing for managing one or more of their patient experience factors. The treatment experience system 100 extracts metadata for the patient selected music or sound representations for patient selected playlist creation. A set of pre-programmed musical sound clips may be selected and prepared by the treatment experience system 100, based on metadata and/or human curation, as representative of a variety of potential music or sound representations that may provide a positive impact for helping Patient manage one or more of their factors. The patient, family or caregiver is presented with an audio survey of the pre-programmed musical sound clips for their preferences. The patient, family or caregivers express their preference from the audio clips, by one of several binary inputs, such as touch [tap, point, click], voice [Yes, No] or gesture [thumb move, or ‘swiping’ images or icons right for ‘like, left for ‘don't like’] of the music or sound representations presented. With appropriate permissions, the treatment experience system 100 can scan the patient, family or caregiver's music library, phone, computer, music storage or streaming services for playlists, likes, preferences, and produce a music audit of the metadata for candidate music or sound representations for patient selected playlists. This playlist creation may provide a positive impact for helping patient manage one or more of their factors [Pain, Stress, Anxiety, Depression, Sleep and Mobility].

The metadata and from the ‘Patient Selected’ pre-programmed musical sound clip preferences, Audio Survey, and Music Audit are used to construct and the Patient Selected Playlist for managing and monitoring, with other data sources such as biometric and/or biochemical feedback, Machine Learning, AI, and the Knowledgebase, for one or more of the Patient Factors being managed [Pain, Stress, Anxiety, Depression, Sleep and Mobility]

When applicable, patients are provided the treatment experience system 100 with both patient selected playlists and the treatment experience system 100 curated Playlists in advance of clinical treatments, such as surgery, dialysis, or scheduled consultations to become familiar with their mind/body control and the biofeedback mechanism for Patient Empowerment and Autonomy to impact their Factor [Pain, Stress, Anxiety, Mobility] with the chosen Complimentary Medicine therapies.

For patients managing chronic conditions, such as lower back pain, or behavioral health conditions such as depression or anxiety, the treatment experience system 100 patient selected playlists and the treatment experience system 100 curated playlists can become a regular monitored part of their overall treatment program for empowerment, autonomy and improvement.

As data is collected by Patient interaction with the treatment experience system 100, the Machine Learning Engine will look for patterns to be discovered by ingesting the data, including, but not limited to the Record Type [Pain, Stress, Anxiety, Depression, Sleep and Mobility], Factors [Self-Reported, Calculated, Biometric], Timestamp, Level, State, Trend and Data Source events, as they vary with the application of a chosen therapy experience and in the case of sound from music, as noted below.

Data patterns and relationships between variables are derived and recognized, using Algorithmic Rules and Machine Learning. They are assessed for the strength and fidelity of the relationship signals in the data, resulting in a degree of certainty assessment for multiple multivariable equations. In essence, the Machine Learning Engine determines the probability that a Factor [Pain] is influenced by the application of an Alternative Therapy [Music]; and specifically, what level of signal specificity for the Music [via Metadata] can be extracted and applied for confirming a Factor Effectiveness Impact Rating index, such as Pain, [+3 to −3, similar to prescription drugs] on the Patient's ability to control their own Pain with lower dosages or no Drugs.

In the Use Case examples above, a Patient was able to achieve a Pain Effectiveness Impact Rating of −3 when listening to the Song ‘Weightless’ by Marconi Union, reducing the need for the available dosage of pain medication. Clinical trials are underway to confirm this preliminary system and method can be scaled up to help millions of Patients now.

By using Complimentary Medicine and the ‘Music and Medicine’ system and method described herein, users can discover Alternative Therapies, targeted to each individual, to biometrically manage Pain and Anxiety, using less drugs, or no drugs at all. Accessing proven alternative therapies for each of the five senses—Sound, Sight, Smell, Touch, Taste, plus Thought, Users measure their individual biometric responses to learn to select the specific alternative therapy(s) that help them control and reduce Pain, Stress & Anxiety.

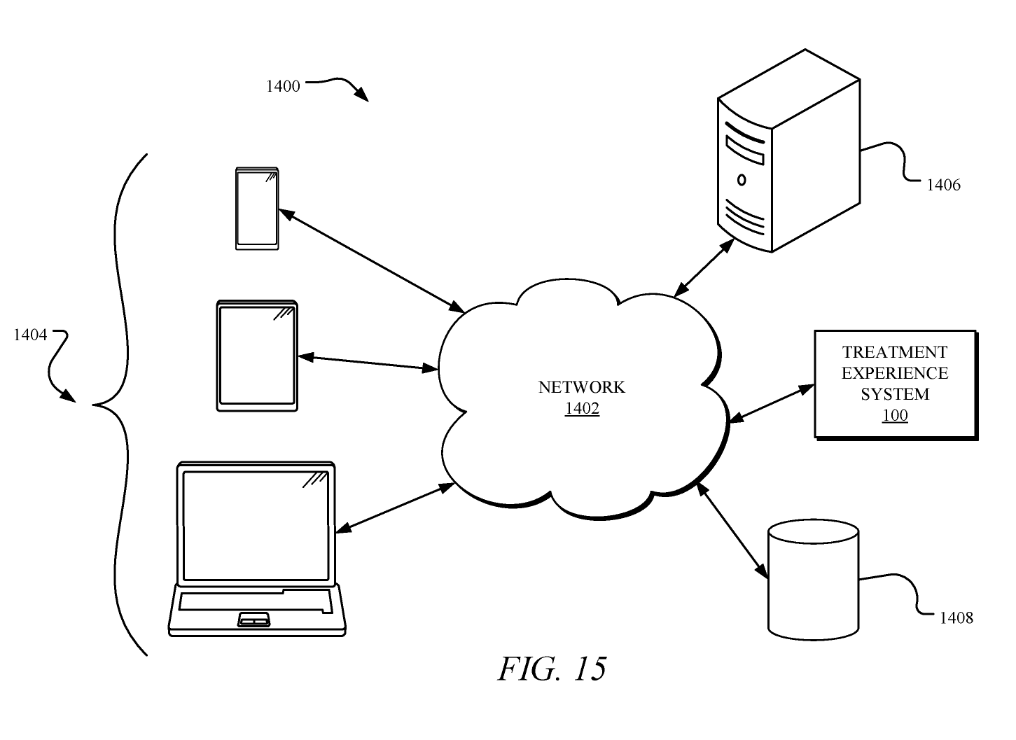

For a detailed description of an example network environment 1400 for generating an optimized treatment experience for a patient, reference is made to FIG. 15. In one implementation, a user, such as a the patient, a care professional, or other authorized party, accesses and interacts with the treatment experience system 100 using a user device 1404 to access or provide an optimized treatment experience or related data via a network 1402.

The user device 1404 is generally any form of computing device capable of interacting with the network 1402, such as the user device 400, one or more of the patient experience components 200, one or more aspects of the treatment experience system 100, or other devices, including, without limitation, a personal computer, terminal, workstation, portable computer, mobile device, smartphone, tablet, multimedia console, and/or the like. The network 1402 is further used by one or more computing or data storage devices (e.g., one or more databases 1408, which may include the intelligence database 212 or other computing units described herein) for implementing the treatment experience system 100 and other services, applications, or modules in the network environment 1400.

In one implementation, the network environment 1400 includes at least one server 1406 hosting a website or an application that the user may visit to access the treatment experience system 100 and/or other network components. The server 1406 may be a single server, a plurality of servers with each such server being a physical server or a virtual machine, or a collection of both physical servers and virtual machines. In another implementation, a cloud hosts one or more components of the network environment 1400. The user devices 1404, the server 1406, and other resources connected to the network 1402 may access one or more other servers to access to one or more websites, applications, web services interfaces, storage devices, computing devices, or the like that are used for patient treatment and/or analysis. The server 1406 may also host a search engine that the treatment experience system 100 uses for accessing, searching for, and modifying patient data, processed therapy data, customized therapies, demographic profiles, non-drug therapeutic indices, demographic therapies, patient management programs, patient intelligence, and other data. In one implementation, the network environment 1400 provides access to resources, therapies, care professionals, data, care intelligence, and/or the like.

In some implementations, users may access and interact with the treatment experience system 100, directly, for example, through the user device 1404 running a browser, application, or other web-service that can interact with the network 1402. In another implementation, the users may access and interact with the treatment experience system 100 from software running on the user device 1404 utilizing an interface such as an application programming interface (API). Stated differently, the API can be called from an application or other software on the user device 1404 to pull or push data to and from the network 1402.

Various information, as described herein may be provided to the one or more databases 1408, which may include a storage cluster or similar storage mechanism configured to parse, tag, and/or associate data elements for storage and analysis. The databases 1408 and/or other components of the treatment experience system 100 may include various modules, components, systems, infrastructures, and/or applications that may be combined in various ways, including into a single software application or multiple software applications. The information provided to the databases 1408 may be stored in one or more non-relational databases and include a distributed, scalable storage layer that is configured to store a large volume of structured and unstructured data. For example, databases 1408 may replicate and distribute blocks of data through cluster nodes, along with numerous other features and advantages. As such, the databases 1408 manage the processing, storage, analysis, and retrieval of large volumes of data in a non-relational database in one implementation.

In some implementations, the databases 1408 and/or other components of the treatment experience system 100 serializes and stores data, such that the patient intelligence for one or more patients, groups of patients, one or more demographics, or groups of demographics may be generated based on a query. The databases 1408 may process a query in multiple parts at a cluster node level and aggregate the results to generate the patient intelligence using the treatment experience system 100. In one implementation, the treatment experience system 100 receives a query in structured query language (SQL), aggregates data stored in the databases 1408, and outputs the patient intelligence in a format enabling further management, analysis, and/or merging with other data sources. The treatment experience system 100 may generate the patient intelligence, patient treatment experiences, and/or other information or conditions using machine learning techniques, as described herein, which generally refers to a machine learning through observing data that represents incomplete information about statistical happenings and generalizing such data to rules and/or algorithms that make predictions for future data, trends, and the like. Machine learning typically includes “classification” where machines learn to automatically recognize complex patterns and make intelligent predictions for a class. The treatment experience system 100 may additionally or alternatively utilize artificial neural networks, deep machine learning, and/or other artificial intelligence techniques to generate the patient treatment experience, patient intelligence, and/or other data. For example, the treatment experience system 100 may obtain input data that changes over time or involves subjective aspects, recognize patterns in the input data, and interpret the patterns through machine perception, labeling, clustering raw input, and/or other clustering, classification, and correlating mechanisms.

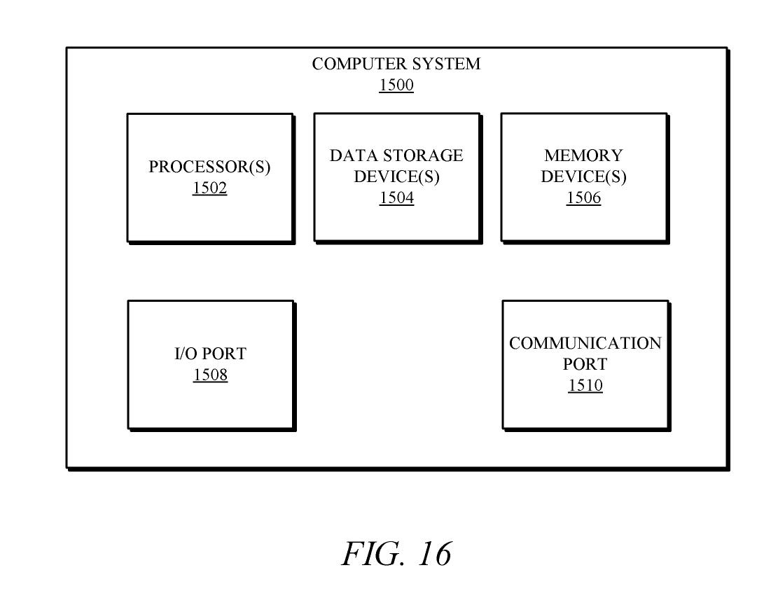

Referring to FIG. 16, a detailed description of an example computing system 1500 having one or more computing units that may implement various systems and methods discussed herein is provided. The computing system 1500 may be applicable to the user devices 400 or 1404, one or more of the patient experience components 200 alone or in some combination, one, more, or all aspects of the treatment experience system 100 alone or in some combination, and other computing or network devices. It will be appreciated that specific implementations of these devices may be of differing possible specific computing architectures not all of which are specifically discussed herein but will be understood by those of ordinary skill in the art.

The computer system 1500 may be a computing system is capable of executing a computer program product to execute a computer process. Data and program files may be input to the computer system 1500, which reads the files and executes the programs therein. Some of the elements of the computer system 1500 are shown in FIG. 16, including one or more hardware processors 1502, one or more data storage devices 1504, one or more memory devices 1508, and/or one or more ports 1508-1510. Additionally, other elements that will be recognized by those skilled in the art may be included in the computing system 1500 but are not explicitly depicted in FIG. 16 or discussed further herein. Various elements of the computer system 1500 may communicate with one another by way of one or more communication buses, point-to-point communication paths, or other communication means not explicitly depicted in FIG. 16.

The processor 1502 may include, for example, a central processing unit (CPU), a microprocessor, a microcontroller, a digital signal processor (DSP), and/or one or more internal levels of cache. There may be one or more processors 1502, such that the processor 1502 comprises a single central-processing unit, or a plurality of processing units capable of executing instructions and performing operations in parallel with each other, commonly referred to as a parallel processing environment.

The computer system 1500 may be a conventional computer, a distributed computer, or any other type of computer, such as one or more external computers made available via a cloud computing architecture. The presently described technology is optionally implemented in software stored on the data stored device(s) 1504, stored on the memory device(s) 1506, and/or communicated via one or more of the ports 1508-1510, thereby transforming the computer system 1500 in FIG. 16 to a special purpose machine for implementing the operations described herein. Examples of the computer system 1500 include personal computers, terminals, workstations, mobile phones, tablets, laptops, personal computers, multimedia consoles, gaming consoles, set top boxes, and the like.

The one or more data storage devices 1504 may include any non-volatile data storage device capable of storing data generated or employed within the computing system 1500, such as computer executable instructions for performing a computer process, which may include instructions of both application programs and an operating system (OS) that manages the various components of the computing system 1500. The data storage devices 1504 may include, without limitation, magnetic disk drives, optical disk drives, solid state drives (SSDs), flash drives, and the like. The data storage devices 1504 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 1506 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 1504 and/or the memory devices 1506, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 1500 includes one or more ports, such as an input/output (I/O) port 1508 and a communication port 1510, for communicating with other computing, network, or vehicle devices. It will be appreciated that the ports 1508-1510 may be combined or separate and that more or fewer ports may be included in the computer system 1500.

The I/O port 1508 may be connected to an I/O device, or other device, by which information is input to or output from the computing system 1500. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 1500 via the I/O port 1508. Similarly, the output devices may convert electrical signals received from computing system 1500 via the I/O port 1508 into signals that may be sensed as output by a human, such as sound, light, and/or touch. The input device may be an alphanumeric input device, including alphanumeric and other keys for communicating information and/or command selections to the processor 1502 via the I/O port 1508. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 1500 via the I/O port 1508. For example, an electrical signal generated within the computing system 1500 may be converted to another type of signal, and/or vice-versa. In one implementation, the environment transducer devices sense characteristics or aspects of an environment local to or remote from the computing device 1500, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 1500, such as, physical movement of some object (e.g., a mechanical actuator), heating or cooling of a substance, adding a chemical substance, and/or the like.

In one implementation, a communication port 1510 is connected to a network by way of which the computer system 1500 may receive network data useful in executing the methods and systems set out herein as well as transmitting information and network configuration changes determined thereby. Stated differently, the communication port 1510 connects the computer system 1500 to one or more communication interface devices configured to transmit and/or receive information between the computing system 1500 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 1510 to communicate one or more other machines, either directly over a point-to-point communication path, over a wide area network (WAN) (e.g., the Internet), over a local area network (LAN), over a cellular (e.g., third generation (3G) or fourth generation (4G)) network, or over another communication means. Further, the communication port 1510 may communicate with an antenna or other link for electromagnetic signal transmission and/or reception.

In an example implementation, patient data, processed therapy data, customized therapies, demographic profiles, non-drug therapeutic indices, demographic therapies, patient management programs, patient intelligence, and software and other modules and services may be embodied by instructions stored on the data storage devices 1504 and/or the memory devices 1506 and executed by the processor 1502. The computer system 1500 may be integrated with or otherwise form part of the treatment experience system 100 in some implementations.

The system set forth in FIG. 16 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

While the present disclosure has been described with reference to various implementations, it will be understood that these implementations are illustrative and that the scope of the present disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A method for generating an optimized treatment experience for a patient, the method comprising:
capturing patient experience data using at least one patient experience device, the patient experience data corresponding to a patient experience factor for the patient;
determining a current level of the patient experience factor using a patient experience processing system, the current level of the patient experience factor determined based on the patient experience data;
generating a customized therapy for the patient based on the current level of the patient experience factor, the customized therapy being an alternative treatment to a drug therapy administration; and
generating an administration of a patient treatment experience based on the customized therapy, the patient treatment experience generated using the at least one patient experience device and including one or more of patient sense stimulation and patient cognitive stimulation.

2. The method of claim 1, further comprising:
capturing patient drug data for the patient; and
determining a current drug administration level for the patient using the patient experience processing system, the current drug administration level determined based on the patient drug data, the customized therapy being further generated based on the current drug administration level.

3. The method of claim 1, further comprising:
capturing biometric response data using at least one patient monitoring device, the biometric response data captured during the administration of the patient experience treatment.

4. The method of claim 3, further comprising:
determining an effectiveness of the customized therapy in addressing the current level of the patient experience factor based on the biometric response data.

5. The method of claim 4, further comprising:
adjusting the patient experience treatment based on the effectiveness of the customized therapy.

6. The method of claim 5, wherein the patient experience treatment is adjusted until the effectiveness includes a target change in the patient experience factor.

7. The method of claim 6, the patient experience treatment includes playing at least one song, a beats per minute of the at least one song being automatically adjusted based on the biometric response data of the patient until the target change is reached.

8. The method of claim 7, wherein the target change includes reaching a target biometric value.

9. The method of claim 7, wherein the biometric response data includes a heart rate of the patient and the target biometric value includes a target heart rate, the beats per minute of the at least one song being adjusted until the heart rate of the patient meets the target heart rate.

10. The method of claim 5, wherein the patient experience treatment is adjusted automatically.

11. The method of claim 4, further comprising:
generating processed therapy data by ingesting one or more of the biometric response data, the customized therapy, the effectiveness of the customized therapy, the patient experience data, and patient drug data.

12. The method of claim 11, further comprising:
generating a baseline for the patient experience factor and a patient profile for the patient using the processed therapy data.

13. The method of claim 12, further comprising:
generating a demographic profile based on the patient profile; and
generating a demographic therapy for the demographic profile using the baseline for the patient experience factor.

14. The method of claim 13, further comprising:
selecting the demographic therapy for a second patient from a plurality of demographic therapies based on a match between the demographic profile with a second patient profile generated for the second patient.

15. The method of claim 14, wherein the demographic therapy is customized for the second patient.

16. The method of claim 4, further comprising:
generating a ranking of the effectiveness of the customized therapy among a plurality of customized therapies for the patient experience factor, the ranking utilized to generate a patient management program for the patient, the patient management program including an aggregated set of the plurality of customized therapies.

17. The method of claim 3, wherein the at least one patient monitoring device includes a biosensor configured to detect one or more biochemicals in saliva of the patient.

18. The method of claim 1, further comprising:
predicting a patient episode based on the current level of the patient experience factor.

19. The method of claim 1, wherein generating the customized therapy includes generating a non-drug therapeutic index for the patient.

20. The method of claim 1, wherein the sense stimulation includes at least one of sight stimulation, taste stimulation, hearing stimulation, touch stimulation, or smell stimulation.

21. The method of claim 1, wherein the customized therapy is further based on one or more historical levels for the patient experience factor.

22. The method of claim 1, wherein the customized therapy includes sound therapy with one or more patient curated playlists.

23. The method of claim 1, wherein the patient experience factor is at least one of pain, stress, anxiety, depression, sleep, or mobility.

24. The method of claim 1, wherein the patient experience data is captured through at least one of: self-reporting by the patient, reporting by a medical professional, facial recognition, gesture recognition, analysis of patient records, or biometric readings.

25. The method of claim 1, wherein the customized therapy is combined with the drug therapy administration for an integrated treatment customized for the patient.
